(19) United States
(12) Patent Application Publication
(10) Pub. No.: US 2009/0258046 A1
(43) Pub. Date: Oct. 15, 2009

(54) COMPOSITION, FORMULATIONS AND KIT FOR TREATMENT OF RESPIRATORY AND LUNG DISEASE WITH NON-GLUCOCORTICOID STEROIDS AND/OR UBIQUINONE AND A BRONCHODILATING AGENT

(76) Inventor: Jonathan W. Nyce, Titusville, NJ (US)

Correspondence Address: WILSON, SONSINI, GOODRICH & ROSATI, 650 PAGE MILL ROAD, PALO ALTO, CA, 94304-1050, US

(21) Appl. No.: 12/430,800

(22) Filed: Apr. 27, 2009

Related U.S. Application Data
(63) Continuation of application No. 10/475,689, filed on Aug. 12, 2004.
(60) Provisional application No. 60/286,139, filed on Apr. 24, 2001.

(51) Int. Cl. A61K 9/00; A61K 31/56; A61K 9/50; A61P 11/00

(52) U.S. Cl. 424/422; 514/171; 424/490

(57) ABSTRACT

A pharmaceutical or veterinary composition, which comprises a first active agent selected from a non-glucocorticoid steroid or analogues, a ubiquinone, or salts thereof, and a second active agent comprising a bronchodilator. The composition is provided in various formulations and in the form of a kit. The products of this patent are applied to the prophylaxis and treatment of respiratory, lung and malignant diseases.

### CROSS REFERENCE

This application is a continuation of the U.S. application Ser. No. 10/475,689, which is a U.S. National Stage Application of the PCT Application No. PCT/US2002/12552, filed on Apr. 22, 2002, which claims priority to the U.S. Provisional Patent Application Ser. No. 60/286,139, filed on Apr. 24, 2001, which are herein incorporated by reference in their entirety.

### BACKGROUND OF THE INVENTION

1. Field of the Invention

This invention relates to a composition and formulations comprising a non-glucocorticoid steroid including DHEA, DHEA salts such as DHEA Sulfate, and analogues and salts thereof, and a bronchodilating agent, and optionally other bioactive agents. These products are useful in the treatment of conditions where a reduction of adenosine levels, or adenosine hyper-responsiveness, or where an increase in ubiquinone or lung surfactant levels is beneficial, or in the treatment of respiratory and lung diseases in general.

2. Description of the Background

Respiratory ailments, associated with a variety of conditions, are extremely common in the general population, and more so in certain ethnic groups, such as African Americans. In some cases they are accompanied by inflammation, which aggravates the condition of the lungs. Asthma, for example, is one of the most common diseases in industrialized countries. In the United States it accounts for about 1% of all health care costs. An alarming increase in both the prevalence and mortality of asthma over the past decade has been reported, and asthma is predicted to be the preeminent occupational lung disease in the next decade. While the increasing mortality of asthma in industrialized countries could be attributable to the depletion reliance upon beta agonists in the treatment of this disease, the underlying causes of asthma remain poorly understood.

Diseases such as asthma, allergic rhinitis, and Acute Respiratory Distress Syndrome (ARDS), including RDS in pregnant mothers and in premature born infants, among others, are common diseases in industrialized countries, and in the United States alone, they account for extremely high health care costs. These diseases have recently been increasing at an alarming rate, both in terms of prevalence, morbidity and mortality. In spite of this, their underlying causes still remain poorly understood.

Asthma is a condition characterized by variable, in many instances reversible obstruction of the airways. This process is associated with lung inflammation and in sum cases lung allergies. Many patients have acute episodes referred to as “asthma attacks,” while others are afflicted with a chronic condition. The asthmatic process is believed to be triggered in some cases by inhalation of antigens by hypersensitive subjects. This condition is generally referred to as “extrinsic asthma.” Other asthmatics have an intrinsic predisposition to the condition, which is thus referred to as “instrinsic asthma,” and may be comprised of conditions of different origin, including those mediated by the adenosine receptor(s), allergic conditions mediated by an immune IgE-mediated response, and others. All asthmas have a group of symptoms, which are characteristic of this condition: bronchoconstriction, lung inflammation and decreased lung surfactant. Existing bronchodilators and anti-inflammatories are currently commercially available and are prescribed for the treatment of asthma. The most common anti-inflammatories, corticosteroids, have considerable side effects but are commonly prescribed nevertheless. Most of the drugs available for the treatment of asthma are, more importantly, barely effective in a small number of patients.

Acute Respiratory Distress Syndrome (ARDS), or stiff lung, shock lung, pump lung and congestive atelectasis, is believed to be caused by fluid accumulation within the lung which, in turn, causes the lung to stiffen. The condition is triggered within 48 hours by a variety of processes that injure the lungs such as trauma, head injury, shock, sepsis, multiple blood transfusions, medications, pulmonary embolism, severe pneumonia, smoke inhalation, radiation, high altitude, near drowning, and others. In general, ARDS occurs as a medical emergency and may be caused by other conditions that directly or indirectly cause the blood vessels to “leak” fluid into the lungs. In ARDS, the ability of the lungs to expand is severely decreased and produces extensive damage to the air sacs and lining or endothelium of the lung. ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. ARDS is commonly diagnosed by testing for symptomatic signs, for example by a simple chest auscultation or examination with a stethoscope that may reveal abnormal symptomatic breath sounds. A preliminary diagnosis of ARDS may be confirmed with chest X-rays and the measurement of arterial blood gas. In some cases ARDS appears to be associated with other diseases, such as acute myelogenous leukemia, with acute tumor lysis syndrome (ATLS) developed after treatment with, e.g. are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

The systemic administration of adenosine was found useful for treating SVT, and as a pharmacologic means to evaluate cardiovascular health via an adenosine stress test commonly administered by hospitals and by doctors in private practice. Adenosine administered by inhalation, however, is known to cause bronchoconstriction in asthmatics, possibly due to mast cell degranulation and histamine release, effects which have not been observed in normal subjects. Adenosine infusion has caused respiratory compromise, for example, in patients with COPD. As a consequence of the untoward side effects observed in many patients, caution is recommended in the prescription of adenosine to patients with a variety of conditions, including obstructive lung disease, emphysema, bronchitis, etc, and complete avoidance of its administration to patients with or prone to bronchoconstriction or bronchospasm, such as asthma. In addition, the administration of adenosine must be discontinued in any patient who develops severe respiratory difficulties. It would be of great help if a formulation were to be made available for joint use when adenosine administration is required.

Allergic rhinitis afflicts one in five Americans, accounting for an estimated $4 to 10 billion in health care costs each year, and occurs at all ages. Because many people mislabel their symptoms as persistent colds or sinus problems, allergic rhinitis is probably underdiagnosed. Typically, IgE combines with allergens in the nose to produce chemical mediators, induction of cellular processes, and neurogenic stimulation, causing an underlying inflammation. Symptoms include nasal congestion, discharge, sneezing, and itching, as well as itchy, watery, swollen eyes. Over time, allergic rhinitis sufferers often develop sinusitis, otitis media with effusion, and nasal polyposis, and may exacerbate asthma, and is associated with mood and cognitive disturbances, fatigue and irritability. Degranulation of mast cells results in the release of preformed mediators that interact with various cells, blood vessels, and mucous glands to produce the typical rhinitis symptoms. Most early- and late-phase reactions occur in the nose after allergen exposure. The late-phase reaction is seen in chronic allergic rhinitis, with hypersecretion and congestion as the most prominent symptoms. Repeated exposure causes a hypersensitivity reaction to one or many allergens. Sufferers may also become hyperreactive to nonspecific triggers such as cold air or strong odors. Nonallergic rhinitis may be induced by infections, such as viruses, or associated with nasal polyps, as occurs in patients with aspirin idiosyncrasy. In addition, pregnancy, hypothyroidism, and exposure to occupational factors or medications can cause rhinitis, as well. NARES syndrome, a non-allergic type of rhinitis associated with eosinophils in the nasal secretions, typically occurs in middle-aged individuals and is accompanied by loss of smell. Saline is often recommended to improve nasal stuffiness, sneezing, and congestion, and saline sprays usually relieve mucosal irritation or dryness associated with various nasal conditions, minimize mucosal atrophy, and dislodge encrusted or thickened mucus, while causing no side effects, and may be tried first in pregnant patients. Also, if used immediately before intranasal corticosteroid dosing, saline helps prevent local irritation. Anti-histamines often serve as a primary therapy. Terfenadine and astemizole, two non-sedating anti-histamines, however, have been associated with a ventricular arrhythmia known as Torsades de Points, usually in interaction with other medications such as ketoconazole and erythromycin, or secondary to an underlying cardiac problem. To date loratadine, another nonsedating anti-histamine, and cetirizine have not been associated with serious adverse cardiovascular events, the most common side effect of cetirizine being drowsiness. Claritin, for example, may be effective in relieving sneezing, runny nose, and nasal, ocular and palatal itching in a low percentage of patients, although not approved for this indication or asthma. Anti-histamines are typically combined with a decongestant to help relieve nasal congestion. Sympathomimetic medications are used as vasoconstrictors and decongestants, the three most common decongestants being pseudoephedrine, phenylpropanolamine and phenylephrine. These agents, however, cause hypertension, palpitations, tachycardia, restlessness, insomnia and headache. Topical decongestants are recommended for a limited period of time, as their overuse results in nasal dilatation. Anti-cholinergic agents, such as Cromolyn, have a role in patients with significant rhinorrhea or for specific entities such as “gustatory rhinitis”, which is usually associated with ingestion of spicy foods, and have been used on the common cold. Sometimes the Cromolyn spray produces sneezing, transient headache, and even nasal burning. Topical and nasal spray corticosteroids such as Vancenase are effective agents in the treatment of rhinitis, especially for symptoms of congestion, sneezing, and runny nose, but often cause irritation, stinging, burning, sneezing, local bleeding and septal perforation. Topical steroids are generally more effective than Cromolyn Sodium, particularly in the treatment of NARES, but side effects limit their usefulness except for temporary therapy in patients with severe symptoms. Immunotherapy, while expensive and inconvenient, often can provide substantial benefits, especially the use of drugs that produce blocking antibodies, alter cellular histamine release, and result in decreased IgE. Presently available treatments, such as propranolol, verapamil, and adenosine, may help to minimize symptoms. Verapamil is most commonly used but it has several shortcomings, since it causes or exacerbates systemic hypotension, congestive heart failure, bradyarrhythmias, and ventricular fibrillation. In addition, verapamil readily crosses the placenta and has been shown to cause fetal bradycardia, heart block, depression of contractility, and hypotension. Adenosine has several advantages over verapamil, including rapid onset, brevity of side effects, theoretical safety, and probable lack of placental transfer, but may not be administered to a variety of patients.

Chronic obstructive pulmonary disease (COPD) is characterized by airflow obstruction that is generally caused by chronic bronchitis, emphysema, or both. Emphysema is characterized by abnormal permanent enlargement of the air spaces distal to the terminal bronchioles, accompanied by destruction of their walls and without obvious fibrosis. Chronic bronchitis is characterized by chronic cough, mucus production, or both, for at least three months for at least two successive years where other causes of chronic cough have been excluded. COPD characteristically affects middle aged and elderly people, and is one of the leading causes of morbidity and mortality worldwide. In the United States it affects about 14 million people and is the fourth leading cause of death. Both morbidity and mortality, however, are rising. The estimated prevalence of this disease in the United States has risen by 41% since 1982, and age adjusted death rates rose by 71% between 1966 and 1985. This contrasts with the decline over the same period in age-adjusted mortality from all causes (which fell by 22%), and from cardiovascular diseases (which fell by 45%). COPD, however, is preventable, since it is believed that its main cause is exposure to cigarette smoke. The disease is rare in lifetime non-smokers, in whom exposure to environmental tobacco smoke will explain at least some of the airways obstruction. Other proposed etiological factors include airway hyper-responsiveness or hypersensitivity, ambient air pollution, and allergy. The airflow obstruction in COPD is usually progressive in people who continue to smoke. This results in early disability and shortened survival time. Stopping smoking reverts the decline in lung function to values for non-smokers. Many patients will use medication chronically for the rest of their lives, with the need for increased doses and additional drugs during exacerbations. Amongst the currently available treatments for COPD, short term benefits, but not long term effects, were found on its progression, from administration of anti-cholinergic drugs, β2 adrenergic agonists, and oral steroids. Neither anti-cholinergic drugs nor β2 adrenergic agonists have an effect on all people with COPD; nor do the two agents combined. The adverse effects of theophyllines and the need for frequent monitoring limit their usefulness. There is no evidence that anti-cholinergic agents affect the decline in lung function, and mucolytics have been shown to reduce the frequency of exacerbations but with a possible deleterious effect on lung function. The long-term effects of β32 adrenergic agonists, oral corticosteroids, and antibiotics have not yet been evaluated, and up to the present time no other drug has been shown to affect the progression of the disease or survival. Thus, there is very little currently available to alleviate symptoms of COPD, prevent exacerbations, preserve optimal lung function, and improve daily living activities a quality of life.

Pulmonary fibrosis, interstitial lung disease (ILD), or interstitial pulmonary fibrosis, include more than 130 chronic lung disorders that affect the lung by damaging lung tissue, and producing inflammation in the walls of the air sacs in the lung, scarring or fibrosis in the interstitium (or tissue between the air sacs), and stiffening of the lung, thus the name of the disease. Breathlessness during exercise may be one of the first symptoms of these diseases, and a dry cough may be present. Neither the symptoms nor X-rays are often sufficient to tell apart different types of pulmonary fibrosis. Some pulmonary fibrosis patients have known causes and some have unknown or idiopathic causes. The course of this disease is generally unpredictable. Its progression includes thickening and stiffening of the lung tissue, inflammation and difficult breathing. Some people may need oxygen therapy as part of their treatment.

Cancer is one of the most prevalent and feared diseases of our times. It generally results from the carcinogenic transformation of normal cells of different epithelia. Two of the most damaging characteristics of carcinomas and other types of malignancies are their uncontrolled growth and their ability to create metastases in distant sites of the host, particularly a human host. It is usually these distant metastases that may cause serious consequences to the host since, frequently, the primary carcinoma is removed by surgery. The treatment of cancer presently relies on surgery, irradiation therapy and systemic therapies such as chemotherapy, different immunity-boosting medicines and procedures, hyperthermia and systemic, radioactively labeled monoclonal antibody treatment, immunotoxins and chemotherapeutic drugs.

Dehydroepiandrosterone (DHEA) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA and DHEA analogues such as DHEA-S sulfate derivative to inhibit carcinogenesis is believed to result from their uncompetitive inhibition of the activity of the enzyme glucose 6-phosphate dehydrogenase (G6PDH). G6PDH is the rate limiting enzyme of the hexose monophosphate pathway, a major source of intracellular ribose-5-phosphate and NADPH. Ribose-5 phosphate is a necessary substrate for the synthesis of both ribo- and deoxyribonucleotides required for the synthesis of RNA and DNA. NADPH is a cofactor also involved in nucleic acid biosynthesis and the synthesis of hydroxmethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultrasensitive to DHEA-mediated NADPH depletion, and that DHEA-treated cells would rapidly show the depletion of intracellular pools of mevalonate. Mevalonate is required for DNA synthesis, and DHEA arrests human cells in the GI phase of the cell cycle in a manner closely resembling that of the direct HMG CoA. Because G6PDH produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is the central precursor for the synthesis of cholesterol, as well as for the synthesis of a variety of non-sterol compounds involved in post-translational modification of proteins such as farnesyl pyrophosphate and geranyl pyrophosphate; for dolichol, which is required for the synthesis of glycoproteins involved in cell-to-cell communication and cell structure; and for ubiquinone, an anti-oxidant with an established role in cellular respiration. It has long been known that patients receiving steroid hormones of adrenocortical origin at pharmacologically appropriate doses show increased incidence of infectious disease.

DHEA, also known as 3β-hydroxyandrost-5-en-17-one or dehydroiso-androsterone, is a 17-ketosteroid which is quantitatively one of the major adrenocortical steroid hormones found in mammals. Although DHEA appears to serve as an intermediary in gonadal steroid synthesis, the primary physiological function of DHEA has not been fully understood. It has been known, however, that levels of this hormone begin to decline in the second decade of life, reaching 5% of the original level in the elderly.) Clinically, DHEA has been used systemically and/or topically for treating patients suffering from psoriasis, gout, hyperlipemia, and it has been administered to post-coronary patients. In mammals, DHEA has been shown to have weight optimizing and anti-carcinogenic effects, and it has been used clinically in Europe in conjunction with estrogen as an agent to reverse menopausal symptoms and also has been used in the treatment of manic depression, schizophrenia, and Alzheimer's disease. DHEA has been used clinically at 40 mg/kg/day in the treatment of advanced cancer and multiple sclerosis. Mild androgenic effects, hirsutism, and increased libido were the side effects observed. These side effects can be overcome by monitoring the dose and/or by using analogues. The subcutaneous or oral administration of DHEA to improve the host's response to infections is known, as is the use of a patch to deliver DHEA. DHEA is also known as a precursor in a metabolic pathway which ultimately leads to more powerful agents that increase immune response in mammals. That is, DHEA acts as a biphasic compound: it acts as an immuno-modulator when converted to androstenediol or androst-5-ene-3β,17β-diol (βAED), or androstenetriol or androst-5-ene-3β7,7β,17β-triol (βAET). However, in vitro DHEA has certain lymphotoxic and suppressive effects on cell proliferation prior to its conversion to βAED and/or βAET. It is, therefore, believed that the superior immunity enhancing properties obtained by administration of DHEA result from its conversion to more active metabolites.

Adequate ubiquinone levels have been found to be essential for maintaining proper cardiac function, and the administration of exogenous ubiquinone has recently been shown to have beneficial effect in patients with chronic heart failure. Ubiquinone depletion has been observed in humans and animals treated with lovastatin, a direct HMG CoA reductase inhibitor. Such lovastatin-induced depletion of ubiquinone has been shown to lead to chronic heart failure, or to a shift from low heart failure into life-threatening high grade heart failure. DHEA, unlike lovastatin, inhibits HMG CoA reductase indirectly by inhibiting G6PDH and depleting NADPH, a required cofactor for HMG CoA reductase. However, DHEA's indirect inhibition of HMG CoA reductase suffices to deplete intracellular mevalonate. This effect adds to the depletion of ubiquinone, and may result in chronic heart failure following long term usage. Thus, although DHEA is considered a safe drug, chronic heart failure may occur as a complicating side effect of its long term administration. Further, some analogues of DHEA produce this side effect to a greater extent because, in general, they are more potent inhibitors of G6PDH than DHEA.

Adenosine is a purine involved in intermediary metabolism, and may constitute an important mediator in the lung for various diseases, including bronchial asthma, COPD, CF, RDS, rhinitis, pulmonary fibrosis, and others. Its potential role was suggested by the finding that asthmatics respond to aerosolized adenosine with marked bronchoconstriction whereas normal individuals do not. An asthmatic rabbit animal model, the dust mite allergic rabbit model for human asthma, responded in a similar fashion to aerosolized adenosine with marked bronchoconstriction whereas non-asthmatic rabbits showed no response. More recent work with this animal model suggested that adenosine-induced bronchoconstriction and bronchial hyperresponsiveness in asthma may be mediated primarily through the stimulation of adenosine receptors. Adenosine has also been shown to cause adverse effects, including death, when administered therapeutically for other diseases and conditions in subjects with previously undiagnosed hyper-reactive airways. Adenosine plays a unique role in the body as a regulator of cellular metabolism. It can raise the cellular level of AMP, ADP and ATP that are the energy intermediates of the cell. Adenosine can stimulate or down regulate the activity of adenylate cyclase and hence regulate cAMP levels. cAMP, in turn, plays a role in neurotransmitter release, cellular division and hormone release. Adenosine's major role appears to be to act as a protective injury autocoid. In any condition in which ischemia, low oxygen tension or trauma occurs adenosine appears to play a role. Defects in synthesis, release, action and/or degradation of adenosine have been postulated to contribute to the over activity of the brain excitatory amino acid neurotransmitters, and hence various pathological states. Adenosine has also been implicated as a primary determinant underlying the symptoms of bronchial asthma and other respiratory diseases, the induction of bronchoconstriction and the contraction of airway smooth muscle. Moreover, adenosine causes bronchoconstriction in asthmatics but not in non-asthmatics. Other data suggest the possibility that adenosine receptors may also be involved in allergic and inflammatory responses by reducing the hyperactivity of the central dopaminergic system. It has been postulated that the modulation of signal transduction at the surface of inflammatory cells influences acute inflammation. Adenosine is said to inhibit the production of super-oxide by stimulated neutrophils. Recent evidence suggests that adenosine may also play a protective role in stroke, CNS trauma, epilepsy, ischemic heart disease, coronary by-pass, radiation exposure and inflammation. Overall, adenosine appears to regulate cellular metabolism through ATP, to act as a carrier for methionine, to decrease cellular oxygen demand and to protect cells from ischemic injury. Adenosine is a tissue hormone or inter-cellular messenger that is released when cells are subject to ischemia, hypoxia, cellular stress, and increased workload, and or when the demand for ATP exceeds its supply. Adenosine is a purine and its formation is directly linked to ATP catabolism. It appears to modulate an array of physiological processes including vascular tone, hormone action, neural function, platelet aggregation and lymphocyte differentiation. It also may play a role in DNA formation, ATP biosynthesis and general intermediary metabolism. It is suggested that it regulates the formation of cAMP in the brain and in a variety of peripheral tissues. Adenosine regulates cAMP formation through two receptors A₁ and A₂. Via A₁ receptors, adenosine reduces adenylate cyclase activity, while it stimulates adenylate cyclase at A₂ receptors. The adenosine A₁ receptors are more sensitive to adenosine than the A₂ receptors. The CNS effects of adenosine are generally believed to be A₁-receptor mediated, where as the peripheral effects such as hypotension, bradycardia, are said to be A₂ receptor mediated.

A handful of medicaments have been used for the treatment of respiratory diseases and conditions, although in general they all have limitations. Amongst them are glucocorticoid steroids, leukotriene inhibitors, anti-cholinergic agents, anti-histamines, oxygen therapy, theophyllines, and mucolytics. Glucocorticoid steroids are the ones with the most widespread use in spite of their well documented side effects. Most of the available drugs are nevertheless effective in a small number of cases, and not at all when it comes to the treatment of asthma. No treatments are currently available for many of the other respiratory diseases. Theophylline, an important drug in the treatment of asthma, is a known adenosine receptor antagonist which was reported to eliminate adenosine-mediated bronchoconstriction in asthmatic rabbits. A selective adenosine A1 receptor antagonist, 8-cyclopentyl-1,3-dipropylxanthine (DPCPX) was also reported to inhibit adenosine-mediated bronchoconstriction and bronchial hyperresponsiveness in allergic rabbits. The therapeutic and preventative applications of currently available adenosine A1 receptor-specific antagonists are, nevertheless, limited by their toxicity. Theophylline, for example, has been widely used in the treatment of asthma, but is associated with frequent, significant toxicity resulting from its narrow therapeutic dose range. DPCPX is far too toxic to be useful clinically. The fact that, despite decades of extensive research, no specific adenosine receptor antagonist is available for clinical use attests to the general toxicity of these agents.

For many years, two classes of compounds have dominated the treatment of asthma: glucocorticosteroids and bronchodilators. Examples of glucocorticosteroids are beclomethasone and corticoid 21-sulfopropionates. Examples of a bronchodilator are an older β2 adrenergic agonist such as albuterol, and a newer one such as salmeterol. In general, when glucocorticosteroids are taken daily either by inhalation or orally, they attenuate inflammation. The β2 adrenergic agonists, on the other hand, primarily alleviate bronchoconstriction. Whereas glucocorticosteroids are not useful in general for acute settings, bronchodilators are used in acute care, such as in the case of asthma attacks. At the present time, many asthma patients require daily use of both types of agents, a glucocorticosteroid to contain pulmonary inflammation, and a bronchodilator to alleviate bronchoconstriction. More recently, fluticasone propionate, a glucocorticoid steroid was combined with β2 adrenergic agonists in one therapeutic formulation said to have greater efficiency in the treatment of asthma. However, glucocorticosteriods, particularly when taken for prolonged periods of time, have extremely deleterious side effects that, although somewhat effective, make their chronic use undesirable, particularly in children.

Clearly, there exists a well defined need for novel and effective therapies for treating respiratory, lung and cancer ailments that cannot presently be treated, or at least for which no therapies are available that are effective and devoid of significant detrimental side effects. This is the case of ailments afflicting the respiratory tract, and more particularly the lung and the lung airways, including respiratory difficulties, bronchoconstriction, lung inflammation and allergies, depletion or hyposecretion of surfactant, etc. Moreover, there is a definite need for treatments that have prophylctic and therapeutic applications, and require low amounts of active agents, which makes them both less costly and less prone to detrimental side effects.

### SUMMARY OF THE INVENTION

The present invention relates to a composition and formulations and treatments employing a first active agent comprising a non-glucocorticoid steroid such as an epiandrosterone (EA) or analogue thereof and/or a ubiquinone (CoEnzyme Q) and/or their salts in combination with a second active agent comprising a bronchodilator, and optionally other bioactive agents and formulation ingredients.

These compositions and formulations are useful for treating lung and respiratory diseases and conditions associated with brochoconstriction, lung inflammation and allergies, and other respiratory and lung diseases.

The drawings accompanying this patent form part of the disclosure of the invention, and further illustrate some aspects of the present invention as discussed below.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates the inhibition of HT-29 SF cells by DHEA.

FIG. 2 illustrates the effects of DHEA on cell cycle distribution in HT-29 SF cells.

FIGS. 3a and 3b illustrate the reversal of DHEA-induced growth inhibition in HT-29 cells.

FIG. 4 illustrates the reversal of DHEA-induced G1 arrest in HT-29 SF cells.

### DETAILED DESCRIPTION OF THE INVENTION

The present invention arose from a desire by the inventor to improve on his prior treatment of respiratory and lung diseases, and other pathologies secondarily afflicting the lung. The present treatment is effective for treating a plurality of diseases, whatever their cause, including steroid administration, abnormalities in adenosine or adenosine receptor metabolism or synthesis, or any other cause. The present invention provides compositions and a method of treating respiratory and lung diseases, whether by reducing adenosine or adenosine receptor levels, reducing hypersensitivity to adenosine, or by increasing ubiquinone or surfactant levels, or any other mechanism, particularly in the lung, liver, heart and brain. The present products are thus indicated for treating diseases and conditions such as respiratory diseases and conditions in general, including asthma, chronic obstructive pulmonary disease (COPD), cystic fibrosis (CF), dispnea, emphysema, wheezing, pulmonary hypertension, pulmonary fibrosis, hyper-responsive airways, increased adenosine or adenosine receptor levels, particularly those associated with infectious diseases, pulmonary bronchoconstriction, lung inflammation and allergies, and surfactant depletion, chronic bronchitis, bronchoconstriction, difficult breathing, impeded and obstructed lung airways, adenosine test for cardiac function, pulmonary vasoconstriction, impeded respiration, Acute Respiratory Distress Syndrome (ARDS), administration of certain drugs, such as adenosine and adenosine level increasing drugs, and other drugs for, e.g. treating SupraVentricular Tachycardia (SVT), and the administration of adenosine stress tests, infantile Respiratory Distress Syndrome (infantile RDS), pain, allergic rhinitis, decreased lung surfactant, decreased ubiquinone levels, or chronic bronchitis, among others.

ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. In the following paragraphs, the specific conditions will be described, and the existing treatments, if any, discussed. ARDS is currently diagnosed by mere symptomatic signs, e.g. chest auscultation with a stethoscope that may reveal abnormal symptomatic breath sounds, and confirmed with chest X-rays and the measurement of arterial blood gas. ARDS, in some instances, appears to be associated with other diseases, such as acute myelogenous leukemia, acute tumor lysis syndrome (ATLS) developed after treatment with, e.g. cytosine arabinoside, etc. In general, however, ARDS is associated with traumatic injury, severe blood infections such as sepsis or other systemic illness, high-dose radiation therapy and chemotherapy, and inflammatory responses which lead to multiple organ failure and in many cases death In premature babies (“premies”), the lungs are not quite developed and, therefore, the fetus is in an anoxic state during development. Moreover, lung surfactant, a material critical for normal respiration, is generally not yet present in sufficient amounts at this early stage of life; however, premies often hyper-express the adenosine A1 receptor and/or underexpress the adenosine A2a receptor and are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

Rhinitis may be seasonal or perennial, allergic or non-allergic. Non-allergic rhinitis may be induced by infections, such as viruses, or associated with nasal polyps, as occurs in patients with aspirin idiosyncrasy. Medical conditions such as pregnancy or hypothyroidism and exposure to occupational factors or medications may cause rhinitis. The so-called NARES syndrome is a non-allergic type of rhinitis associated with eosinophils in the nasal secretions, which typically occurs in middle-age and is accompanied by some loss of sense of smell. When cholinergic pathways are stimulated they produce typical secretions that are identified by their glandular constituents so as to implicate neurologic stimulation. Other secretions typical of increased vascular permeability are found in allergic reactions as well as upper respiratory infections, and the degranulation of mast cells releases preformed mediators that interact with various cells, blood vessels, and mucous glands, to produce the typical rhinitis symptoms. Most early- and late-phase reactions occur in the nose after allergen exposure. The late-phase reaction is seen in chronic allergic rhinitis, with hypersecretion and congestion as the most prominent symptoms. When priming occurs, it exhibits a lowered threshold to stimulus after repeated allergen exposure which, in turn, causes a hypersensitivity reaction to one or more allergens. Sufferers may also become hyper-reactive to non-specific triggers such as cold air or strong odors. Self-administered saline improves nasal stuffiness, sneezing, and congestion and usually causes no side effects and it is, thus, the first treatment tried in pregnant patients. Saline sprays are generally used to relieve mucosal irritation or dryness associated with various nasal conditions, minimize mucosal atrophy, and dislodge encrusted or thickened mucus. If used immediately before intranasal corticosteroid dosing, saline sprays may help prevent drug-induced local irritation. Anti-histamines such as terfenadine and astemizole, two non-sedating anti-histamines, are also employed to treat this condition, but have been associated with a ventricular arrhythmia known as Torsades de Points, usually in interaction with other medications such as ketoconazole and erythromycin, or secondary to an underlying cardiac problem. Loratadine, another non-sedating anti-histamine, and cetirizine have not been associated with an adverse impact on the QT interval, or with serious adverse cardiovascular events. Cetirizine, however, produces extreme drowsiness and has not been widely prescribed. Non-sedating anti-histamines, e.g. Claritin, may produce some relieving of sneezing, runny nose, and nasal, ocular and palatal itching, but have not been tested for asthma or other more specific conditions. Terfenadine, loratadine and astemizole, on the other hand, exhibit extremely modest bronchodilating effects, reduction of bronchial hyper-reactivity to histamine, and protection against exercise- and antigen-induced bronchospasm Some of these benefits, however, require higher-than-currently-recommended doses. The sedating-type anti-histamines help induce night sleep, but they cause sleepiness and compromise performance if taken during the day. When employed, anti-histamines are typically combined with a decongestant to help relieve nasal congestion. Sympathomimetic medications are used as vasoconstrictors and decongestants. The three commonly prescribed systemic decongestants, pseudoephedrine, phenylpropanolamine and phenylephrine cause hypertension, palpitations, tachycardia, restlessness, insomnia and headache. The interaction of phenylpropanolamine with caffeine, in doses of two to three cups of coffee, may significantly raise blood pressure. In addition, medications such as pseudoephedrine may cause hyperactivity in children. Topical decongestants, nevertheless, are only indicated for a limited period of time, as they are associated with a rebound nasal dilatation with overuse. Anti-cholinergic agents are given to patients with significant rhinorrhea or for specific conditions such as “gustatory rhinitis”, usually caused by ingestion of spicy foods, and may have some beneficial effects on the common cold. Cromolyn, for example, if used prophylactically as a nasal spray, reduces sneezing, rhinorrhea, and nasal pruritus, and blocks both early- and late-phase hypersensitivity responses, but produces sneezing, transient headache, and even nasal burning. Topical corticosteroids such as Vancenase are somewhat effective in the treatment of rhinitis, especially for symptoms of congestion, sneezing, and runny nose. Depending on the preparation, however, corticosteroid nose sprays may cause irritation, stinging, burning, or sneezing, as well. Local bleeding and septal perforation can also occur sometimes, especially if the aerosol is not aimed properly. Topical steroids generally are more effective than cromolyn sodium, particularly in the treatment of NARES, and also to reduce some symptoms of rhinitis. Their side effects, however, limit their usefulness except for temporary therapy in patients with severe symptoms. These agents are sometimes used for shrinking nasal polyps when local therapy fails. Immunotherapy, while expensive and inconvenient, often provides benefits, especially for inpatients who experience side effects from other medications. So-called blocking antibodies, and agents that alter cellular histamine release, eventually result in decreased IgE, along with many other favorable physiologic changes. This effect is useful in IgE-mediated diseases, e.g., hypersensitivity in atopic patients with recurrent middle ear infections. For allergic rhinitis sufferers, however, a runny nose is more than a nuisance. The disorder often results in impaired quality of life and sets the stage for more serious ailments, including psychological problems. Presently, rhinitis is mostly treated with propranolol, verapamil, and adenosine, all of which have Food and Drug Administration-approved labeling for acute termination of supraventricular tachycardia (SVT).

There is very little currently available to alleviate symptoms of COPD, prevent exacerbations, preserve optimal lung function, and improve daily living activities and quality of life. Anti-cholinergic drugs achieve short-term bronchodilation and produce some symptom relief in people with COPD, but no improved long-term prognosis even with inhaled products. Most COPD patients have at least some measure of airways obstruction that is somewhat alleviated by ipratropium bromide. “The lung health study” found in men and women smokers spirometric signs of early COPD. Three treatments compared over a five year period found that ipratropium bromide had no significant effect on the decline in the functional effective volume of the patient's lungs whereas smoking cessation produced a slowing of the decline in the functional effective volume of the lungs. Ipratropium bromide, however, produced serious adverse effects, such as cardiac symptoms, hypertension, skin rashes, and urinary retention. Short and long acting inhaled β2 adrenergic agonists achieve short-term bronchodilation and provide some symptomatic relief in COPD patients, but show no meaningful maintenance effect on the progression of the disease. Short acting β2 adrenergic agonists improve symptoms in subjects with COPD, such as increasing exercise capacity and produce some degree of bronchodilation, and even an increase in lung function in some severe cases. The maximum effectiveness of the newer long acting inhaled β2 adrenergic agonists was found to be comparable to that of short acting β2 adrenergic agonists. Salmeterol was found to improve symptoms and quality of life, although only producing modest or no change in lung function. In asthmatics, however, β2 adrenergic agonists have been linked to an increased risk of death, worsened control of asthma, and deterioration in lung function. Continuous treatment of asthmatic and COPD patients with the bronchodilators, ipratropium bromide or fenoterol resulted in a faster decline in lung function, when compared with treatment provided on a need basis, therefore indicating that they are not suitable for maintenance treatment. The most common immediate adverse effect of β2 adrenergic agonists, on the other hand, is tremors, which at high doses may cause a fall in plasma potassium, dysrhythmias, and reduced arterial oxygen tension. The combination of a β2 adrenergic agonist with an anti-cholinergic drug provides little additional bronchodilation compared with either drug alone. The addition of ipratropium to a standard dose of inhaled β2 adrenergic agonists for about 90 days, however, produces some improvement in stable COPD patients over either drug alone. Anti-cholinergic agents were found to produce greater bronchodilation with anti-cholinergic agents than β2 adrenergic agonists in people with COPD. Ipratropium bromide given to patients without bronchodilator therapy produced an improvement of the functional effective volume of the patient's lungs that was greater when administered in conjunction with an anti-cholinergic agent than with a β2 adrenergic agonist, given the residual effect of the anti-cholinergic drug. Overall, the occurrence of adverse effects with β2 adrenergic agonists, such as tremor and dysrhythmias, is more frequent than with anti-cholinergics. Theophyllines have a small bronchodilatory effect in COPD patients whereas they have some common adverse effects, and they have a small therapeutic range given that blood concentrations of 15-20 mg/l are required for optimal effects. Adverse effects include nausea, diarrhea, headache, irritability, seizures, and cardiac arrhythmias, and they occur at highly variable blood concentrations and, in many people, they occur within the therapeutic range. The theophyllines' doses must be adjusted individually according to smoking habits, infection, and other treatments, which is cumbersome. Although theophyllines have been claimed to have an anti-inflammatory effect in asthma, especially at lower doses, none has been reported in COPD, although their bronchodilating short-term effect appears to be statistically different from placebo. Oral corticosteroids show some improvement in baseline functional effective volume in stable COPD patients whereas systemic corticosteroids have been found to be harmful at least producing some osteoporosis and inducing overt diabetes. The longer term use of oral corticosteroids may be useful in COPD, but it usefulness must be weighed against their substantial adverse effects. Inhaled corticosteroids have been found to have no real short-term effect in airway hyper-responsiveness to histamine, but a small long-term effect on lung function, e.g.; in pre-bronchodilator functional effective volume. Fluticasone treatment of COPD patients showed a significant reduction in moderate and severe (but not mild) exacerbations, and a small but significant improvement in lung function and six minute walking distance. Oral prednisolone, inhaled beclomethasone or both had no effects in COPD patients, but lung function improved oral corticosteroids. Mucolytics have a modest beneficial effect on the frequency and duration of exacerbations but an adverse effect on lung function. Neither N-acetylcysteine nor other mucolytics, however, have a significant effect in people with severe COPD (functional effective volume <50%) in spite of evidencing greater reductions in frequency of exacerbation. N-acetylcysteine produced gastrointestinal side effect. Long-term oxygen therapy administered to hypoxaemic COPD and congestive cardiac failure patients, had little effect on their rates of death for the first 500 days or so, but survival rates in men increased afterwards and remained constant over the next five years. In women, however, oxygen decreased the rates of death throughout the study. Continuous oxygen treatment of hypoxemic COPD patients (functional effective volume <70% predicted) for 19.3 years decreased overall risk of death. To date, however, only life style changes, smoking cessation and long term treatment with oxygen (in hypoxaemics), have been found to alter the long-term course of COPD.

Although the progress and symptoms of pulmonary fibrosis and other ILDs may vary from person to person, they have one common link: they affect parts of the lung. When inflammation involves the walls of the bronchioles (small airways), it is called bronchiolitis, when it involves the walls and air spaces of the alveoli (air sacs), it is called alveolitis, and when it involves the small blood vessels (capillaries) of the lungs, it is called vasculitis. The inflammation may heal, or it may lead to permanent scarring of the lung tissue, in which case it is called pulmonary fibrosis. This fibrosis or scarring of the lung tissue results in permanent loss of its ability to breathe and carry oxygen, and the amount of scarring determines the level of disability a person experiences because of the destruction by the scar tissue of the air sacs and lung tissue between and surrounding the air sacs and the lung capillaries. When this happens, oxygen is generally administered to help improve breathing. Pulmonary fibrosis is caused by, or takes the form of, occupational and environmental exposure to irritants such as asbestos, silica and metal dusts, bacteria and animal dusts, gases and fumes, asbestosis and silicosis, infections that produce lung scarring, of which tuberculosis is one example, connective tissue or collagen diseases such as Rheumatoid Arthritis, Systemic Sclerosis and Systemic Lupus Erythematosis, idiopathic pulmonary fibrosis and, although not as common, pulmonary fibrosis of genetic/familial origin and certain medicines. Many of the diseases are often named after the occupations with which they are associated, such as Grain handler's lung, Mushroom worker's lung, Bagassosis, Detergent worker's lung, Maple bark stripper's lung, Malt worker's lung, Paprika splitter's lung, and Bird breeder's lung. “Idiopathic” (of unknown origin) pulmonary fibrosis (IPF) is the label applied when all other causes of interstitial lung disease have been ruled out, and is said to be caused by viral illness and allergic or environmental exposure (including tobacco smoke). Bacteria and other microorganisms are not thought to be a cause of IPF. There is also a familial form of the disease, known as familial idiopathic pulmonary fibrosis whose main symptom is shortness of breath. Since many lung diseases show this symptom, making a correct diagnosis is often difficult. The shortness of breath may first appear during exercise and the condition may progress then to the point where any exertion is impossible, eventually resulting in shortness of breath even at rest. Other symptoms may include a dry cough (without sputum), and clubbing of the fingertips. Glucocorticosteroids are usually administered to treat inflammation present in pulmonary fibrosis, with inconclusive results. Other drugs, however, are not usually added until it is clear that the steroids are not effective in reversing the disease. Glucocorticosteroids are also used in combination with other drugs when a diagnosis is first established, for example oxygen therapy prescribed in severe cases.

The administration of influenza and pneumococcal pneumonia vaccines is often recommended in pulmonary fibrosis and more generally for all lung diseases to prevent infection. The treatment and management of pulmonary fibrosis often requires a lung biopsy to assess the unpredictable response of patients to glucocorticosteroids or other immune system suppressants. Lung transplants are sometimes an ultimate option in severe cases of pulmonary fibrosis and other lung diseases. Pulmonary fibrosis may also be caused by other specific diseases, such as sarcoidosis, a disease whose cause is unknown, that is characterized by the formation of granulomas or areas of inflammatory cells. The disease may attack any organ of the body, but most frequently attacks the lungs, and is generally diagnosed when a chest x-ray shows enlarged lymph glands in the center of both lungs or evidence of lung tissue thickening. For many sarcoidosis is a minor problem, and symptoms including dry cough, shortness of breath, mild chest pain, fatigue, weakness and weight loss-may appear infrequently and stop even without medication. For others, it is a serious, disabling disease that affects African-americans more than members of any other race, although almost everybody may develop the disease, most common in young adults 20 to 40. Histiocytosis X, also associated with pulmonary fibrosis, seems to begin in the bronchioles or small airways of the lungs and their associated arteries and veins, and is generally followed by destruction of the bronchioles and narrowing and damaging of small blood vessels. It is diagnosed by a bronchoalveolar lavage test involving the removal and identification of cells from the lower respiratory tract. Symptoms of this disease include a dry cough (without sputum), breathlessness upon exertion, and/or chest pain. In approximately 50% of the cases, the disease is chronic with loss of lung function, and although glucocorticosteroid therapy is often prescribed, there is no evidence that it is effective. Many histiocytosis X sufferers are current or former cigarette smokers, although its association with smoking is not well understood. Many jobs, particularly those that involve mining or that expose workers to asbestos or metal dusts, may cause pulmonary fibrosis by inhalation of small particulate matter, e.g. dust or asbestos fibers that damage the lungs, especially the small airways and air sacs, and cause scarring (fibrosis). Agricultural workers are also affected by some particulate organic substances, such as moldy hay, which cause an allergic reaction in the lung called “Farmer's Lung”, and may cause pulmonary fibrosis as well.

Asbestosis and silicosis are two occupational lung diseases whose causes are known. Asbestosis is caused by small needle-like particles of asbestos inhaled into the lungs, and cause lung scarring or pulmonary fibrosis that may lead to lung cancer. Silicosis is a dust disease that comes from breathing in free crystalline silica dust, and is produced by all types of mining in which the ore, e.g. gold, lead, zinc, copper, iron, anthracite (hard) coal, and some bituminous (soft) coal, are extracted from quartz rock. Workers in foundries, sandstone grinding, tunneling, sandblasting, concrete breaking, granite carving, and china manufacturing also encounter silica. Large silica particles are stopped in the upper airways, but the tiniest specks of silica are carried down to the lung alveoli, where they lead to pulmonary fibrosis. The use of glucocorticosteroids alone, or combined drug therapy, and the hope of lung transplant are three treatment approaches that are currently being tested, but up to the present time there is no good therapy for this disease. This patent provides the first effective therapy for these and other respiratory and lung ailments.

In the present context, the terms “adenosine”, “surfactant” and “ubiquinone” depletion are intended to encompass levels are lowered or depleted in the subject as compared to previous levels in that subject, and levels that are essentially the same as previous levels in that subject but, because of some other reason, a therapeutic benefit would be achieved in the patient by modification of the levels of these agents as compared to previous levels.

To be administered to a subject are a first active agent selected from an epiandrosterone, analogues or their pharmaceutically or veterinarily acceptable salts and/or a ubiquinone (CoQₙ, where n=1-12) or its pharmaceutically or veterinarily acceptable salts, and a second active agent selected from bronchodilators such as β-2 adrenergic agonists, anti-cholinergic agents, anti-histamines, leukotriene inhibitors, theophyllines, and mucolytics, among others. The first and second agents are administered in therapeutic or prophylactic amounts that are effective to inhibit, delay or control the treated diseases, particularly those associated with lung vasoconstriction, bronchoconstriction, inflammation, allergies, fibrosis, cancerous tissue and others benefiting from adenosine depletion or a reversal of ubiquinone and lung surfactant depletion. More specifically, in one embodiment the pharmaceutical or veterinary composition of the invention comprises a first active agent selected from non-glucocorticoid steroid having the chemical formula I

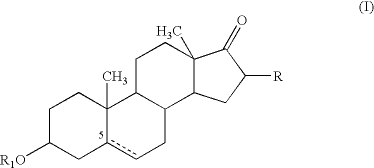

wherein the broken line represents a single or a double bond; R is hydrogen or a halogen; the H at position 5 is present in the alpha or beta configuration or the compound of chemical formula I comprises a racemic mixture of both configurations; and R₁ is hydrogen or SO₂OM, wherein M is selected from the group consisting of H, Na, sulfatide

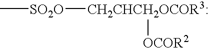

and phosphatide 3

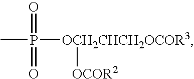

wherein R² and R³, which may be the same or different, are straight or branched (C₁-C₁₄) alkyl or glucuronide

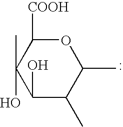

non-glucocorticoid steroids of the chemical formula

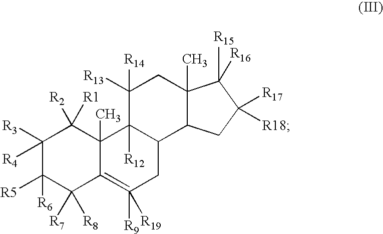

non-glucocorticoid steroid of the chemical formula

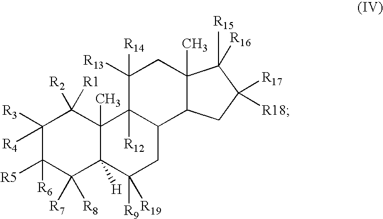

wherein R1, R2, R3, R4, R5, R7, R8, R9, R10, R12, R13, R14 and R19 are independently H, OR, halogen (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothiane, —OSO2R20, —OPOR20R21 or (C1-C10) alky, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H halogen, (C1-C10) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R20 or —OPOR20R21 when R16 is H, or R15 and R16 taken together are ═O; R17 and R18 are independently (1) H, —OH, halogen, (C1-C10) alkyl or —(C1-C10) alkoxy when R6 is H OR, halogen. (C1-C10) alkyl or —C(O)OR22, (2) H, (C1-C10 alkyl).amino, ((C1-C10) alkyl)n amino-(C1-C10) alkyl, (C1-C10) alkoxy, hydroxy —(C1-C10) alkyl, (C1-C10) alkoxy —(C1-C10) alkyl, (halogen)m (C1-C10) alkyl, (C1-C10) alkanoyl, formyl, (C1-C10) carbalkoxy or (C1-C10) alkanoyloxy when R15 and R16 taken together are ═O, (3) R17 and R18 taken together are ═O; (4) R17 or R18 taken together with the carbon to which they are attached form a 3-6 member ring containing 0 or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)m (C1-C10) alkyl or (C1-C10) alkyl; n is 0, 1 or 2; and m is 1, 2 or 3; or pharmaceutically or veterinarily acceptable salts thereof; and/or a ubiquinone and pharmaceutically or veterinarily acceptable salt thereof, wherein the ubiquinone has the chemical formula

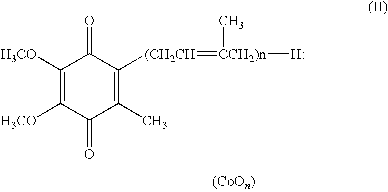

wherein n=1 to 12 and a second active agent comprising an agent; wherein the first agent is present in an amount effective for one or more of altering levels of, or sensitivity to, adenosine, or increasing levels of ubiquinone or lung surfactant, in a subject's tissue(s), or for preventing or treating a respiratory disease.

The hydrogen atom at position 5 of the chemical formula I may be present in the alpha or beta configuration, or the DHEA compound may be provided as a mixture of compounds of both configurations.

Compounds illustrative of chemical formula I above are included, although not exclusively, are DHEA, wherein R and R¹ are each hydrogen, containing a double bond; 16-alpha bromoepiandrosterone, wherein R is Br, R¹ is H, containing a double bond; 16-alpha-fluoro epiandrosterone, wherein R is F, R¹ is H, containing a double bond; Etiocholanolone, wherein R and R¹ are each hydrogen lacking a double bond; and Dehydroepiandrosterone sulphate, wherein R is H, R¹ is SO₂OM and M is a sulphatide group as defined above, lacking a double bond. Others, however, are also included. Also preferred compounds of formula I are those where R is halogen, e.g. bromo, chloro, or fluoro, where R1 is hydrogen, and where the double bond is present. A most preferred compound of formula I is 16-alpha-fluoro epiandrosterone. Other preferred compounds are DHEA and DHEA salts, such as the sulfate salt (DHEA-S). Other DHEA analogues and derivatives suitable for use in this invention are non-glucocorticoid steroid of the chemical formula

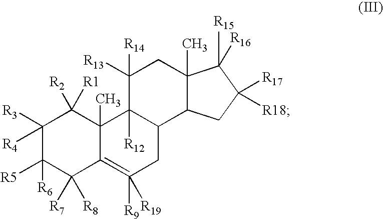

or a non-glucocorticoid steroid of the chemical formula

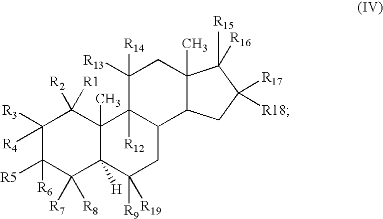

wherein R1, R2, R3, R4, R5, R7, R8, R9, R10, R12, R13, R14 and R19 are independently H, OR halogen, (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20, —OPOR20R21 or (C1-C10) alky, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H, halogen, (C1-C10) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20 or —OPOR20R21 when R16 is H, or R15 and R16 taken together are ═O; R17 and R18 are independently (1) H, —OH, halogen, (C1-C10) alkyl or —(C1-C10) alkoxy when R6 is H OR, halogen. (C1-C10) alkyl or —C(O)OR22, (2) H, (C1-C10 alkyl).amino, ((C1-C10) alkyl)n amino-(C1-C10) alkyl, (C1-C10) alkoxy, hydroxy —(C1-C10) alkyl, (C1-C10) alkoxy —(C1-C10) alkyl, (halogen)m (C1-C10) alkyl, (C1-C10) allanoyl, formyl, (C1-C10) carbalkoxy or (C1-C10) alkanoyloxy when R15 and R16 taken together are ═O, (3) R17 and R18 taken together are ═O; (4) R17 or R18 taken together with the carbon to which they are attached form a 3-6 member ring contain or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)m (C1-C10) alkyl or (C1-C10) alkyl; n is 0, 1 or 2; and m is 1, 2 or 3; or pharmaceutically or veterinarily acceptable salts thereof Of the non-glucocorticoid steroids of formulas (III) and (IV), preferred are those where R15 and R16 together are ═O, also preferred are those where R5 is OH, where R5 is —SO2R2O, and where R20 is H.

In general, the non-glucocorticoid steroid, such as those of formulas (I), (III) and (IV), their derivatives and their salts are administered in a dosage of about 0.05, about 0.1, about 1, about 5, about 20 to about 100, about 500, about 1000, about 1500 about 1,800, about 2500, about 3000, about 3600 mg/kg body weight. Other dosages, however, are also suitable and are contemplated within this patent. The first active agent of formula I, III and IV may be made in accordance with known procedures, or variations thereof that will be apparent to those skilled in the art. See, for example, U.S. Pat. No. 4,956,355; UK Patent No. 2,240,472; EPO Patent Application No. 429; 187, PCT Patent Publication No. WO 91/04030; U.S. Pat. No. 5,859,000; Abou-Gharbia et al., J. Pharm. Sci. 70: 1154-1157 (1981); Merck Index Monograph No. 7710 (11th Ed. 1989), among others.

The other first active agent, the ubiquinone, is a naturally occurring substance, which is also available commercially. The ubiquinone may be administered with the second agent, a bronchodilator, such as a β2 adrenergic agonist, and optionally a non-glucocorticoid steroid of formula (I), (II), (I) or (IV), or a glucocorticosteroid and/or other bioactive agents, separately and concurrently, before or after one another, or in the same composition. Among the other bioactive agents, preferred is the administration of the ubiquinone with folinic acid and/or its salts. The phrase “concurrently administering”, as used herein, means that the ubiquinone or its salt is administered either simultaneously in time (preferably by formulating the two together in a common pharmaceutical carrier), or at different times during the course of a common treatment schedule. In the case where both DHEA and Ubiquinone are administered, they may be administered at times sufficiently close so that the ubiquinone, in addition to its direct effect, may off-set any detrimental effects of agents, such as on ubiquinone levels, e.g. in the lungs and heart of the subject, and thereby counter-balance any deterioration of function that may result from its administration. The term “ubiquinone or CoEnzyme Q”, as used herein, refers to a family of compounds having structures based on a 2,3-dimethoxy-5-methyl benzoquinone nucleus with a variable terpenoid acid chain containing one to twelve non-unsaturated trans-isoprenoid units. Such compounds are known in the art as “CoEnzyme Qₙ”, wherein n is 1 to 12. These compounds may be referred to herein as compounds represented by the formula

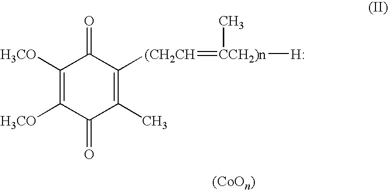

In the method of the invention, the ubiquinone is preferably a compound according to the chemical formula given above, wherein n=1-10, more preferably n=6-10, i.e. Coenzymes Q₆₋₁₀, and most preferably n=10, i.e. Coenzyme Q₁₀. The ubiquinone and the non-glucocorticoid steroid or EA or their salts may be formulated with a pharmaceutically acceptable carrier, alone or with the second active agent, but separately from the other first active agent or salt thereof As in the case where the non-glucocorticoid steroid, or salt thereof, is either not being administered or it is administered directly to the lungs of the subject, the ubiquinone may be administered systemically. The composition may be formulated by any of the techniques set forth in this patent and others as an artisan would know.

In general, the ubiquinone is administered in a therapeutic amount for treating the targeted disease or condition, and/or an amount effective to off-set ubiquinone depletion or maintain healthy levels of ubiquinone in the lungs and heart of the subject, and the dosage will vary depending upon the condition of the subject, other agents being administered, the type of formulation employed, and the route of administration. The ubiquinone is preferably administered in a total amount per day of about 0.1, about 1, about 3, about 5, about 10, about 15, about 30 to about 50, about 100, about 150, about 300, about 600, about 900, about 1200 mg/kg body weight. More preferred are about 1 to about 150 mg/kg, about 30 to about 100 mg/kg, and most preferred about 5 to about 50 mg/kg. Other amounts may, of course, be employed as well in accordance with the state of the patient, other agents administered and route of administration, as an artisan would know. The ubiquinone may be administered once or several times a day.

The non-glucocorticoid steroid, ubiquinone, bronchodilators, and other drugs used to treat respiratory, lung and neoplastic diseases, and any of the additional agents listed below, may be administered per se or in the form of pharmaceutically acceptable salts, as discussed above, all being referred to as “active compounds or agents.” The present active agents may also be administered in combination with one another, in the form of separate, or jointly in, pharmaceutically or veterinarily acceptable formulation(s). The active compounds or their salts may be administered either systemically or topically, as discussed below.

Examples of bronchodilating agents are ubiquinones, glucocorticoids, adenosine receptor antagonists such as theophyllines, anti-cholinergics, and β2 adrenergic agonists.

Examples of β2 adrenergic agonists are ephedrine, isoproterenol, isoetharine, epinephrine, metaproterenol, terbutaline, fenoterol, procaterol, albuterol, salmeterol, pirbuterol, formoterol, biloterol, bambuterol, salbutamol and seretide, among others. Examples of glucocorticosteroids, such as beclomethasone, corticoid 21-sulfopropionates, (16 alpha)-16,17-alkylidenebis(oxy)-3-arylpregna-2,4-trien-20-ones, hydroco esters, cyproterone thiopivalate (CTP), hydrocortisone, dexamethasone trimethyl acetate, alkane sulfonic acids of decinine, α-hydroxyprednisolone, 18,18-difluorosteroids, preparing 17α-hydroxy corticoid 21-phosphate, 21-phosphate corticords having unprotected hydroxyl radicals at least at the 17α- and 21-position, 16α-methylated δ-17(20)-corticoids, 21-(L-ascorbyl-2-phosphor-yl)dexamethasone, 21-(L ascorbyl-2-phosphoryl)hydrocortisone, 21-(L-ascorbyl-2-phosphoryl)triamcinolone acetonide and physiologically acceptable salts thereof, among others. Some of these are effective for short periods of time, but in conjunction with the non-glucocorticoid steroids provide a good combination of short and long term relief.

The daily dosage of the bronchodilators and the optional anti-inflammatory glucocorticosteroid to be administered to a subject will vary with the overall treatment programmed, the agent employed, the type of formulation, the route of administration and the state of the patient. A large number of bronchodilators and anti-inflammatory glucocorticosteroids are known in the art, and are commercially available. Their use is widespread and their broad range of dosages is in the public domain. See, e.g. U.S. Pat. No. 5,270,350 for salmeterol. Examples 11 to 21 show aerosolized preparations in accordance with the invention for delivery with a device for respiratory or nasal administration, or administration by inhalation. For intrapulmonary administration, liquid preparations are preferred. In the case of other bioactive agents, there exist FDA recommended amounts for supplementing a person's dietary intake with additional bioactive agents, such as in the case of vitamins and minerals. However, where employed for the treatment of specific conditions or for improving the immune response of a subject they may be utilized in dosages hundreds and thousands of times higher. Mostly, the pharmacopeia's recommendations cover a very broad range of dosages, from which the medical artisan may draw guidance. Amounts for the exemplary agents described in this patent may be in the range of those currently being recommended for daily consumption, below or above those levels. The treatment may typically begin with a low dose of a bronchodilator in combination with a non-glucocorticoid steroid or a ubiquinone, and optionally a glucocorticoid steroid, or other bioactive agents as appropriate, and then a titration up of the dosage for each patient Higher and smaller amounts, including initial amounts, however, may be administered within the confines of this invention as well.

Preferable ranges for the first, second and other agents employed here will vary depending on the route of administration and type of formulation employed, as an artisan will appreciate and manufacture in accordance with known procedures and components. The active compounds may be administered as one dose (once a day) or in several doses (several times a day). The compositions and method of preventing and treating respiratory, cardiac, cardiovascular and neoplastic diseases, among others, including aging, may be used to treat adults and infants, as well as non-human animals afflicted with the described conditions. Although the present invention is concerned primarily with the treatment of human subjects, it may also be employed, for veterinary purposes in the treatment of other mammalian subjects, such as dogs and cats as well as for large domestic and wild animals. The terms “high” and “low” levels of “adenosine” and “adenosine receptors” as well as “adenosine depletion” are intended to encompass both, conditions where adenosine levels are higher than, or lower (even depleted) when compared to previous adenosine levels in the same subject, and conditions where adenosine levels are within the normal range but, because of some other condition or alteration in that patient, a therapeutic benefit would be achieved in the patient by decreasing or increasing adenosine or adenosine receptor levels or hypersensitivity. Thus, this treatment helps regulate (titrate) the patient in a custom tailored manner. Whereas the administration of an agent such as non-glucocorticoid steroid or ubiquinone in accordance with this invention, may decrease or even deplete adenosine levels in a subject having either normal or high levels prior to treatment, the further administration of a bronchodilator will improve the subject's respiration in a short period of time. Ubiquinones themselves, however, also have bronchodilating activity. The further addition of other therapeutic agents will help titrate undesirably low levels of adenosine, which may be observed upon the administration of the present treatment, particularly until an optimal titration of the appropriate dosages is attained.

Other agents that may be incorporated into the present composition are one or more of a variety of therapeutic agents that are administered to humans and animals. Some of the categories of agents suitable for incorporation into the present composition and formulations are analgesics, pre-menstrual medications, menopausal agents, anti-aging agents, anti-anxyolytic agents, mood disorder agents, anti-depressants, anti-bipolar mood agents, anti-schyzophrenic agents, anti-cancer agents, alkaloids, blood pressure controlling agents, hormones, anti-inflammatory agents, muscle relaxants, steroids, soporific agents, anti-ischemic agents, anti-arrythmic agents, contraceptives, vitamins, minerals, tranquilizers, neurotransmitter regulating agents, wound healing agents, anti-angyogenic agents, cytokines, growth factors, anti-metastatic agents, antacids, anti-histaminic agents, anti-bacterial agents, anti-viral agents, anti-gas agents, appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, analgesics, pre-menstrual medications, anti-menopausal agents such as hormones and the like, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, hormones, anti-inflammatory agents, other agents suitable for the treatment and prophylaxis of diseases and conditions associated or accompanied with pain and inflammation, such as arthritis, burns, wounds, chronic bronchitis, chronic obstructive pulmonary disease (COPD), inflammatory bowel disease such as Crohn's disease and ulcerative colitis, autoimmune disease such as lupus erythematosus, muscle relaxants, steroids, soporific agents, anti-ischemic agents, anti-arrhythmic agents, contraceptives, vitamins, minerals, tranquilizers, neurotransmitter regulating agents, wound and burn healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, etc.

Among the hormones are female and male sex hormones such as premarin, progesterone, androsterones and their analogues, thyroxine and glucocorticoids, among the libido altering agents are Viagra and other NO-level modulating agents, among the analgesics are over-the-counter medications such as ibuprofen, oruda, aleve and acetaminofen and controlled substances such as morphine and codeine, among the anti-depressants are tricyclics, MAO inhibitors and epinephrine, .gamma.-amino butyric acid (GABA), dopamine and serotonin level elevating agents, e.g. Prozac, Amytryptilin, Wellbutrin and Zoloft, among the skin renewal agents are Retin-A, hair growth agents such as Rogaine, among the anti-inflammatory agents are non-steroidal anti-inflammatory drugs (NSAIDs) and steroids, among the soporifics are melatonin and sleep inducing agents such as diazepam, cytoprotective, anti-ischemic and head injury agents such as enadoline, and many others. Examples of agents in the different groups are provided in the following list. Examples of analgesics are Acetominophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Examples of anti-anxiety agents include Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Examples of anti-anxiety agents associated with mental depression are Chlordiazepoxide, Amitriptyline, Loxapine Maprotiline and Perphenazine, among others. Examples of anti-inflammatory agents are non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin. Examples of anti-inflammatories for ocular treatment are Diclofenac, Flurbiprofen, Indomethacin, Ketorolac, Rimexolone (generally for post-operative treatment). Examples of anti-inflammatories for non-infectious nasal applications are Beclomethaxone, Budesonide, Dexamethasone, Flunisolide, Triamcinolone, and the like. Examples of soporifics (anti-insomnia/sleep inducing agents) such as those utilized for treatment of insomnia, are Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Examples of sedatives are Diphenhydramine, Hydroxyzine, Methotrimeprazine, Promethazine, Propofol, Melatonin, Trimeprazine, and the like. Examples of sedatives and agents used for treatment of petit mal and tremors, among other conditions, are Amitriptyline HCl, Chlordiazepoxide, Amobarbital, Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia) include Enadoline HCl (e.g. for treatment of severe head injury, orphan status, Warner Lambert). Examples of cytoprotective agents and agents for the treatment of menopause and menopausal symptoms are Ergotanine, Belladonna Alkaloids and Phenobarbitals. Examples of agents for the treatment of menopausal vasomotor symptoms are Clonidine, Conjugated Estrogens and Medroxyprogesterone, Estradiol, Estradiol Cypionate, Estradiol Valerate, Estrogens, conjugated Estrogens, esterified Estrone, Estropipate and Ethinyl Estradiol. Examples of agents for treatment of symptoms of Pre Menstrual Syndrome (PMS) are Progesterone, Progestin, Gonadotrophic Releasing Hormone, oral contraceptives, Danazol, Luprolide Acetate and Vitamin B6. Examples of agents for the treatment of emotional/psychiatric treatments are Tricyclic Antidepressants including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications. Examples of anti-migraine agents are Imitrex and the like.

The active agents of this invention are provided within broad amounts of the composition. For example, the active agents may be contained in the composition in amounts of about 0.001%, about 1%, about 2%, about 5%, about 10%, about 20%, about 40%, about 90%, about 98%, about 99.999% of the composition. The amount of each active agent may be adjusted when, and if, additional agents with overlapping activities are included as discussed in this patent. The dosage of the active compounds, however, may vary depending on age, weight, and condition of the subject. Treatment may be initiated with a small dosage, e.g. less than the optimal dose, of the first active agent of the invention, be it a non-glucocorticoid steroid or a ubiquinone, and optionally other bioactive agents described above. This may be similarly done with the second active agent, until a desirable level is attained. Or vice versa, for example in the case of multivitamins and/or minerals, the subject may be stabilized at a desired level of these products and then administered the first active compound. The dose may be increased until a desired and/or optimal effect under the circumstances is reached. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, the dose of one of the other or of both agents may be adjusted to attain a desirable effect without exceeding a dose range that avoids untoward side effects. Thus, for example, when other analgesic and anti-inflammatory agents are added to the composition, they may be added in amounts known in the art for their intended application or in doses somewhat lower that when administered by themselves.

Pharmaceutically acceptable salts should be pharmacologically and pharmaceutically or veterinarily acceptable, and may be prepared as alkaline metal or alkaline earth salts, such as sodium, potassium or calcium salts. Organic salts and esters are also suitable for use with this invention. The active compounds are preferably administered to the subject as a pharmaceutical or veterinary composition, which includes systemic and topical formulations. Among these, preferred are formulations suitable for inhalation, or for respirable, buccal, oral, rectal, vaginal, nasal, intrapulmonary, ophthalmic, optical, intracavitary, intratraccheal, intraorgan, topical (including buccal, sublingual, dermal and intraocular), parenteral (including subcutaneous, intradermal, intramuscular, intravenous and intraarticular) and tnansdermal administration, among others. The compositions may conveniently be presented in single or multiple unit dosage forms as well as in bulk, and may be prepared by any of the methods which are well known in the art of pharmacy. The composition of the invention may also be provided in the form of a kit whether already formulated or where the active agents are separately provided along with other ingredients, and instructions for its formulation and administration regime. The kit may also contain other agents, such as those described in this patent and, for example, when for parenteral administration, they may be provided with a carrier in a separate container, where the carrier may be sterile. The present composition may also be provided in lyophilized form, and in a separate container, which may be sterile, for addition of a liquid carrier prior to administration. See, e.g. U.S. Pat. No. 4,956,355; UK Patent No. 2,240,472; EPO Patent Application Serial No. 429, 187; PCT Patent Publication WO 91/04030; Mortensen, S. A., et al., Int. J. Tiss. Reac. XII(3): 155-162 (1990); Greenberg, S. et al., J. Clin. Pharm 30: 596-608 (1990); Folkers, K., et al., P. N. A. S. (USA) 87: 8931-8934 (1990), the relevant preparatory and compound portions of which are incorporated by reference above.

The present composition is provided in a variety of systemic and topical formulations. The systemic or topical formulations of the invention are selected from the group consisting of oral, intrabuccal, intrapulmonary, rectal, intrauterine, intradermal, topical, dermal, parenteral, intratumor, intracranial, intrapulmonary, buccal, sublingual, nasal, intramuscular, subcutaneous, intravascular, intrathecal, inhalable, respirable, transdermal, intraarticular, intracavitary, implantable, transdermal, iontophoretic, intraocular, ophthalmic, vaginal, optical, intravenous, intramuscular, intraglandular, intraorgan, intralymphatic, implantable, slow release and enteric coating formulations. The actual preparation and compounding of these different formulations is known in the art and need not be detailed here. The active compounds may be administered once or several times a day.

Formulations suitable for respiratory, nasal, intrapulmonary, and inhalation administration are preferred, as are topical, oral and parenteral formulations. All methods of preparation include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and a suitable carrier. In general, the compositions of the invention are prepared by uniformly and intimately admixing the active compound with a liquid or finely divided solid carrier, or both, and then, if necessary, shaping the resulting mixture. For example, a tablet may be prepared by compressing or molding a power or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispensing agent(s). Molded tablets may be made by molding, in a suitable machine, the powdered compound moistened with an inert liquid binder. A syrup may be made by adding the active compound to a concentrated aqueous solution of a sugar, for example sucrose to which may also be added any accessory ingredient(s). Such accessory ingredient(s) may include flavorings, suitable preservatives, an agent to retard crystallization of the sugar, and an agent to increase the solubility of any other ingredient, such as a polyhydric alcohol, for example glycerol or sorbitol. Compositions for oral administration may optionally include enteric coatings known in the art to prevent degradation of the compositions in the stomach and provide release of the drug in the small intestine. Compositions suitable for buccal or sub-lingual administration include lozenges comprising the active compound in a flavored base, usually sucrose and acacia or tragacanth and pastilles comprising the compound in an inert base such as gelation and glycerin or sucrose and acacia.

Compositions suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Extemporaneous injection solutions and suspensions may be prepared from sterile powders, granules and tablets of the kind previously described.

Nasal and instillable formulations comprise purified aqueous solutions of the active compound with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye. Optical formulations are generally prepared in viscous carriers, such as oils and the like, as is known in the art, so that they may be easily administered into the ear without spilling.

Compositions suitable for topical application to the skin preferably take the form of an ointment, cream, lotion, paste, gel, spray, aerosol, or oil. Carriers which may be used include Vaseline, lanolin, polyethylene glycols, alcohols, transdermal enhancers, and combinations of two or more thereof. Compositions suitable for transdermal administration may be presented as discrete patches adapted to remain in intimate contact with the epidermis of the recipient for a prolonged period of time. Compositions suitable for transdermal administration may also be delivered by iontophoresis. See, for example, Pharmaceutical Research 3:318 (1986), and typically take the form of an optionally buffered aqueous solution of the active compound. Topical formulations comprise the active compound dissolved or suspended in one or more media such as mineral oil, petroleum, polyhydroxy alcohols or other bases used for topical pharmaceutical formulations. Cosmetic formulations may be in the form of solid or liquid preparations, for spreading on a subject's skin, including skin base, pancake, suntan, self-tanning and sun blocking lotions and oils. These formulations may additionally contain other cosmetic ingredients as are known in the art. Examples of these formulations are lotions, creams, oils, and other ointments, e.g. suntan lotions containing sunscreens and other protective ingredients, facial make-up and cleansing formulations, shampoos, hair and skin conditioners, and many more known in the art and commercially available. The addition of other accessory ingredients, vide infra, may be desirable, for example, accessory ingredient(s) selected from diluents, buffers, flavoring, coloring and aromatizing agents, binders, disintegrants, surface active agents, thickeners, lubricants, emulsifiers, surfactants, emollients, preservatives (including anti-oxidants), and the like. Other ingredients may also be utilized as is known in the art.

The active compounds disclosed herein may be administered into the respiratory system either by inhalation, respiration, nasal administration or intrapulmonary instillation (into the lungs) of a subject by any suitable means, and are preferably administered by generating an aerosol or spray comprised of powdered or liquid nasal, intrapulmonary, respirable or inhalable particles. The respirable or inhalable particles comprising the active compound are inhaled by the subject, i.e, by inhalation or by nasal administration or by instillation into the respiratory tract or the lung itself. The formulation may comprise respirable or inhalable liquid or solid particles of the active compound that, in accordance with the present invention, include respirable or inhalable particles of a size sufficiently small to pass through the mouth and larynx upon inhalation and continue into the bronchi and alveoli of the lungs. In general, particles ranging from about 0.05, about 0.1, about 0.5, about 1, about 2 to about 4, about 6, about 8, about 10 microns in size. More particularly, about 0.5 to less than about 5 microns in size, are respirable or inhalable. Particles of non-respirable size which are included in an aerosol or spray tend to deposit in the throat and be swallowed. The quantity of non-respirable particles in the aerosol is, thus, preferably minimized. For nasal administration or intrapulmonary instillation, a particle size in the range of about 8, about 10, about 20, about 25 to about 35, about 50, about 100, about 150, about 250, about 500 μm is preferred to ensure retention in the nasal cavity or for instillation and direct deposition into the lung. Liquid formulations may be squirted into the respiratory tract (nose) and the lung, particularly when administered to newborns and infants.

Liquid pharmaceutical compositions of active compound for producing an aerosol may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant that serves to facilitate the formation of an aerosol. A suitable dispersant is lactose, which may be blended with the active compound in any suitable ratio, e.g., a 1 to 1 ratio by weight.

Aerosols of liquid particles comprising the active compound may be produced by any suitable means, such as with a nebulizer. See, e.g. U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w composition, but preferably less than 20% w/w carrier being typically water or a dilute aqueous alcoholic solution, preferably made isotonic with body fluids by the addition of, for example sodium chloride. Optional additives include preservatives if the composition is not prepared sterile, for example, methyl hydroxybenzoate, anti-oxidants, flavoring agents, volatile oils, buffering agents and surfactants. Aerosols of solid particles comprising the active compound may likewise be produced with any sold particulate medicament aerosol generator. Aerosol generators for administering solid particulate medicaments to a subject product particles which are respirable, as explained above, and generate a volume of aerosol containing a predetermined metered dose of a medicament at a rate suitable for human administration. Examples of such aerosol generators include metered dose inhalers and insufflators.

Having now generally described this invention, the same will be better understood by reference to certain specific examples, which are included herein for purposes of illustration only and are not intended to be limiting of the invention or any embodiment thereof, unless so specified.

### EXAMPLES

In the following examples, DHEA means dehydroepiandrosterone, s means seconds, mg means milligrams, kg means kilograms, kw means kilowatts, Mhz means megahertz, and nmol means nanomoles.

### Examples 1 and 2

### In Vivo Effects of Folinic Acid & DHEA on Adenosine Levels

Young adult male Fischer 344 rats (120 grams) were administered dehydroepiandrosterone (DHEA) (300 mg/kg) or methyltestosterone (40 mg/kg) in carboxymethylcellulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen with 10 seconds of death. Liver and lungs were removed en bloc and flash frozen with 30 seconds of death. Brain tissue was subsequently dissected. Tissue adenosine was extracted, derivatized to 1,N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 1 below. Results are expressed as the mean±SEM, with κ ρ<0.05 compared to control group and φ ρ<0.05 compared to DHEA or methyltestosterone-treated groups.

TABLE 1
In vivo Effect of DHEA, δ-1-methyltestosterone &
Folinic Acid on Adenosine Levels in various Rat Tissues

| | Intracellular adenosine (nmols)/mg protein | | | |
|---|---|---|---|---|
| Treatment | Heart | Liver | Lung | Brain |
| Control | 10.6 ± 0.6 | 14.5 ± 1.0 | 3.1 ± 0.2 | 0.5 ± 0.04 |
| | (n = 12) | (n = 12) | (n = 6) | (n = 12) |
| DHEA | 6.7 ± 0.5 | 16.4 ± 1.4 | 2.3 ± 0.3 | 0.19 ± 0.01 |
| (300 mg/kg) | (n = 12) | (n = 12) | (n = 6) | (n = 12) |
| Methyl- | 8.3 ± 1.0 | 16.5 ± 0.9 | N.D. | 0.42 ± 0.06 |
| testosterone | (n = 6) | (n = 6) | | (n = 6) |
| (40 mg/kg) | | | | |
| Methyl- | 6.0 ± 0.4 | 5.1 ± 0.5 | N.D. | 0.32 ± 0.03 |
| testosterone | (n = 6) | (n = 6) | | (n = 6) |
| (120 mg/kg) | | | | |
| Folinic Acid | 12.4 ± 2.1 | 16.4 ± 2.4 | N.D. | 0.72 ± 0.09 |
| (50 mg/kg) | (n = 5) | (n = 5) | | (n = 5) |
| | (n = 5) | (n = 5) | | (n = 5) |
| DHEA | 11.1 ± 0.6 | 18.8 ± 1.5 | N.D. | 0.55 ± 0.09 |
| (300 mg/kg) + | (n = 5) | (n = 5) | | (n = 5) |
| Folinic Acid | | | | |
| (50 mg/kg) | | | | |
| Methyl- | 9.1 ± 0.4 | N.D. | N.D. | 0.60 ± 0.06 |
| testosterone | (n = 6) | | | (n = 6) |
| (120 mg/kg) + | | | | |
| Folinic Acid | | | | |
| (50 mg/kg) | | | | |

N.D. = Not Determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Coadministration of folinic acid completely abrogated steroid-mediated adenosine depletion. Folinic acid administered alone induce increase in adenosine levels for all organs studied.

### Example 3

### Preparation of the Experimental Model

Cell cultures, HT-29 SF cells, which represent a subline of HY-29 cells (ATCC, Rockville, Md.) and are adapted for growth in completely defined serum-free PC-1 medium (Ventrex, Portland, Me.), were obtained. Stock cultures were maintained in this medium at 37° (in a humidified atmosphere containing 5% CO₂). At confluence cultures were replated after dissociation using trypsin/EDTA (Gibco, Grand Island, N.Y.) and re-fed every 24 hours. Under these conditions, the doubling time for HT-29 SF cells during logarithmic growth was 24 hours.

### Example 4

### Flow Cytometry

Cells were plated at 10⁵/60-mm dish in duplicate. For analysis of cell cycle distribution, cultures were exposed to either 0, 25, 50, or 200 μM DHEA. For analysis of reversal of cell cycle effects of DHEA, cultures were exposed to either 0 or 25 μM DHEA, and the media were supplemented with MVA, CH, RN, MVA plus CH, or MVA plus CH plus RN or were not supplemented. Cultures were trypsinized following 0, 24, 48, or 74 hours and fixed and stained using a modification of a procedure of Bauer et al., Cancer Res., 46, 3173-3178 (1986). Briefly, cells were collected by centrifugation and resuspended in cold phosphate-buffered saline. Cells were fixed in 70% ethanol, washed, and resuspended in phosphate-buffered saline. One ml hypotonic stain solution [50 μg/1 ml propidium iodide (Sigma Chemical Co.), 20 μg/ml Rnase A (Boehringer Mannheim, Indianapolis, Ind.), 30 mg/ml polyethylene glycol, 0.1% Triton X-100 in 5 mM citrate buffer] was then added, and after 10 min at room temperature, 1 ml of isotonic stain solution [propidium iodide, polyethylene glycol, Triton X-100 in 0.4M NaCl] was added and the cells were analyzed using a flow cytometer, equipped with pulse width/pulse area doublet discrimination (Becton Dickinson Immunocytometry Systems, San Jose, Calif.) After calibration with fluorescent beads, a minimum of 2×10⁴ cells/sample were analyzed, data were displayed s total number of cells in each of 1024 channels of increasing fluorescence intensity, and the resulting histogram was analyzed using the Cellfit analysis program (Becton Dickinson).

### Example 5

### DHEA Effect on Cell Growth

Cells were plated 25,000 cells/30 mm dish in quadruplicate, and after 2 days received 0, 12.5, 25, 50, or 200 μM DHEA. Cell number was determined 0, 24, 48, and 72 hours later using a Coulter counter (model Z7 Coulter Electronics, Inc. Hialeah, Fla.). DHEA (AKZO, Basel, Switzerland) was dissolved in dimethyl sulfoxide, filter sterilized, and stored at −20° C. until use.

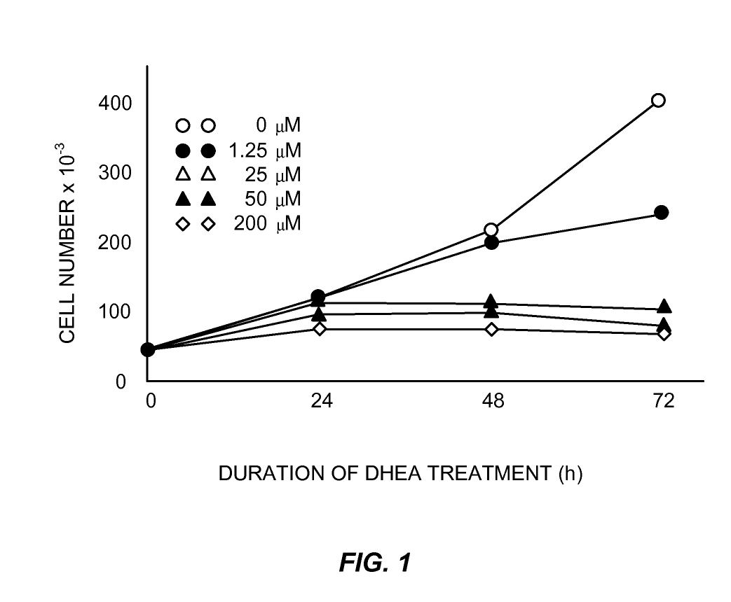

FIG. 1 illustrates the inhibition of growth for HT-29 cells by DHEA. Points refer to numbers of cells, and bars refer to SEM. Each data point was performed in quadruplicate, and the experiment was repeated three times. Where SEM bars are not apparent, SEM was smaller than symbol. Exposure to DHEA resulted in a reduced cell number compared to controls after 72 hours in 12.5 μM, 48 hours in 25 or 50 μM, and 24 hours in 200 μM DHEA, indicating that DHEA produced a time- and dose-dependent inhibition of growth.

### Example 6

### DHEA Effect on Cell Cycle

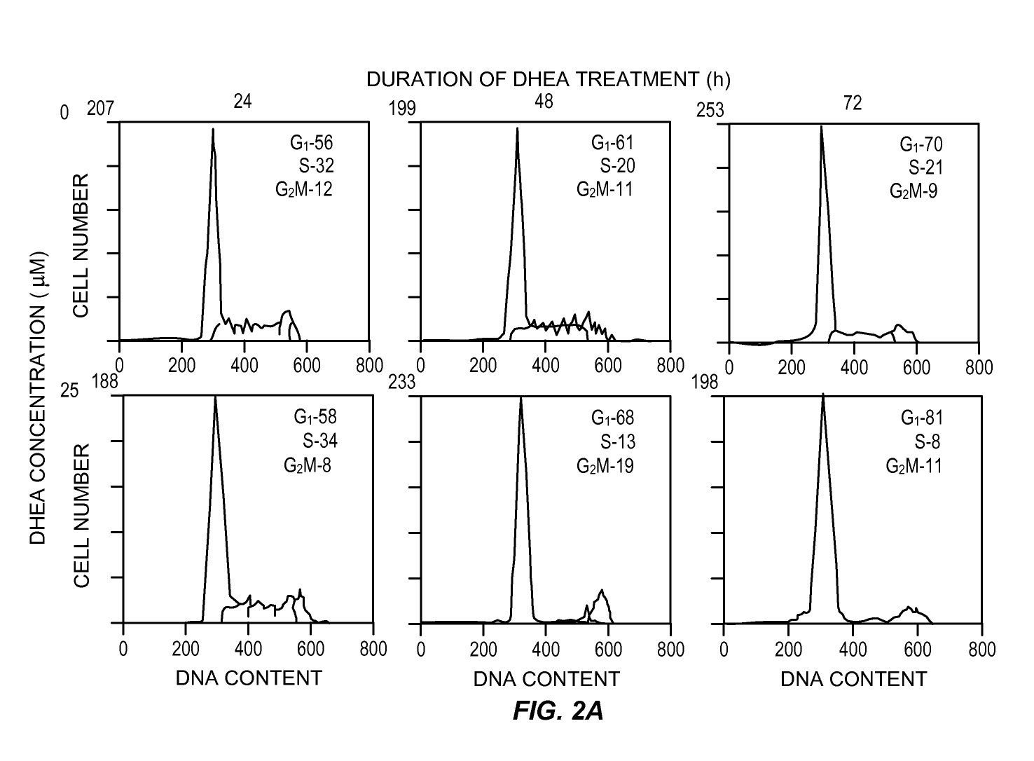

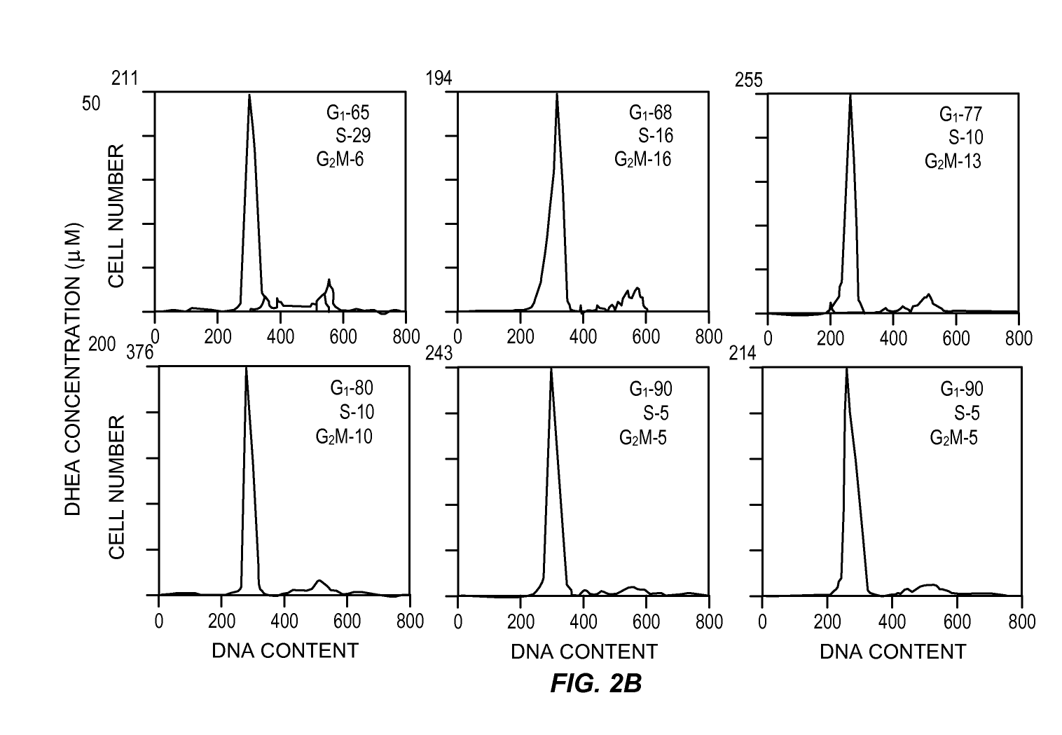

To examine the effects of DHEA on cell cycle distribution, HT-29 SF cells were plated (10⁵ cells/60 mm dish), and 48 hours later treated with 0, 25, 50, or 200 μM DHEA. FIG. 2 illustrates the effects of DHEA on cell cycle distribution in HT-29 SF cells. After 24, 48, and 72 hours, cells were harvested, fixed in ethanol, and stained with propidium iodide, and the DNA content/cell was determined by flow cytometric analysis. The percentage of cells in G₁, S, and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased, and the percentage of cells in G₁, S and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased and the percentage of cells in G₁ phase was increased after 72 hours. A transient increase in G₂M phase cells was apparent after 48 hours. Exposure to 200 μM DHEA produced a similar but more rapid increase in the percentage of cells in G₁ and a decreased proportion of cells in S phase after 24 hours, which continued through the treatment. This indicates that DHEA produced a G₁ block in HT-29 SF cells in a time- and dose-dependent manner.

### Example 7

### Reversal of DHEA-Mediated Effect on Growth & Cell Cycle

Reversal of DHEA-mediated Growth Inhibition. Cells were plated as above, and after 2 days received either 0 or 25 μM DHEA-containing medium supplemented with mevalonic acid (“MVA”; mM) squalene (SQ; 80 μM), cholesterol (CH; 15 μg/ml), MVA plus CH, ribonucleosides (RN; uridine, cytidine, adenosine, and guanosine at final concentrations of 30 μM each), deoxyribonucleosides (DN; thynidine, deoxycytidine, deoxyadenosine and deoxyguanosine at final concentrations of 20 μM each). RN plus DN, or MVA plus CH plus RN, or medium that was not supplemented. All compounds were obtained from Sigma Chemical Co. (St. Louis, Mo.) Cholesterol was solubilized in ethanol immediately before use. RN and DN were used in maximal concentrations shown to have no effects on growth in the absence of DHEA.

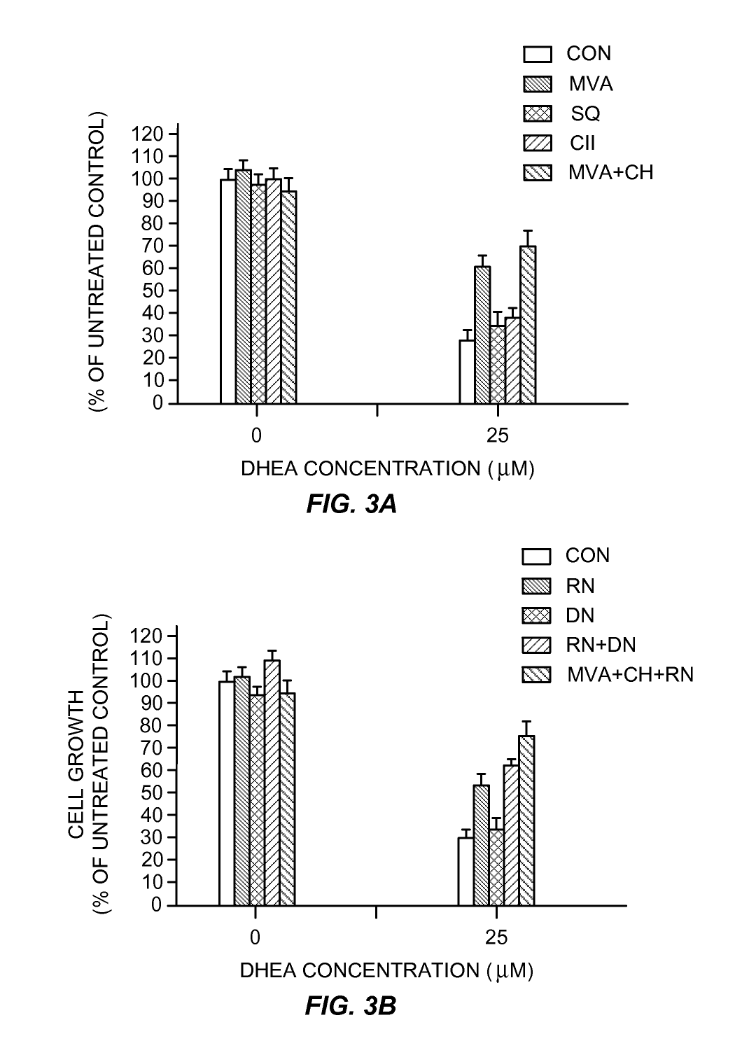

FIG. 3 illustrates the reversal of DHEA-induced growth inhibition in HT-29 SF cells. In A, the medium was supplemented with 2 μM MVA, 80 μM SQ, 15 μg/ml CH, or MVA plus CH (MVA+CH) or was not supplemented (CON). In B, the medium was supplemented with a mixture of RN containing uridine, cytidine, adenosine, and guanosine in final concentrations of 30 μM each; a mixture of DN containing thymidine, deoxycytidine, deoxyadenosine and deoxyguanosine in final concentrations of 20 μM each; RN plus DN (RN+DN); or MVA plus CH plus RN (MVA+CH+RN). Cell numbers were assessed before and after 48 hours of treatment, and culture growth was calculated as the increase in cell number during the 48 hour treatment period. Columns represent cell growth percentage of untreated controls; bars represent SEM. Increase in cell number in untreated controls was 173, 370″6518. Each data point represents quadruplicate dishes from four independent experiments. Statistical analysis was performed using Student's t test κ ρ<0.01; φ ρ<, 0.001; compared to treated controls. Note that supplements had little effect on culture growth in absence of DHEA.

Under these conditions, the DHEA-induced growth inhibition was partially overcome by addition of MVA as well as by addition of MVA plus CH. Addition of SQ or CH alone had no such effect. This suggest that the cytostatic activity of DHEA was in part mediated by depletion of endogenous mevalonate and subsequent inhibition of the biosynthesis of an early intermediate in the cholesterol pathway that is essential for cell growth. Furthermore, partial reconstitution of growth was found after addition of RN as well as after addition of RN plus DN but not after addition of DN, indicating that depletion of both mevalonate and nucleotide pools is involved in the growth-inhibitory action of DHEA. However, none of the reconstitution conditions including the combined addition of MVA, CH, and RN completely overcame the inhibitory action of DHEA, suggesting either cytotoxic effects or possibly that additional biochemical pathways are involved.

### Example 8

### Reversal of DHEA Effect on Cell Cycle

HT-29 SF cells were treated with 25 FM DHEA in combination with a number of compounds, including MVA, CH, or RN, to test their ability to prevent the cell cycle-specific effects of DHEA. Cell cycle distribution was determined after 48 and 72 hours using flow cytometry.

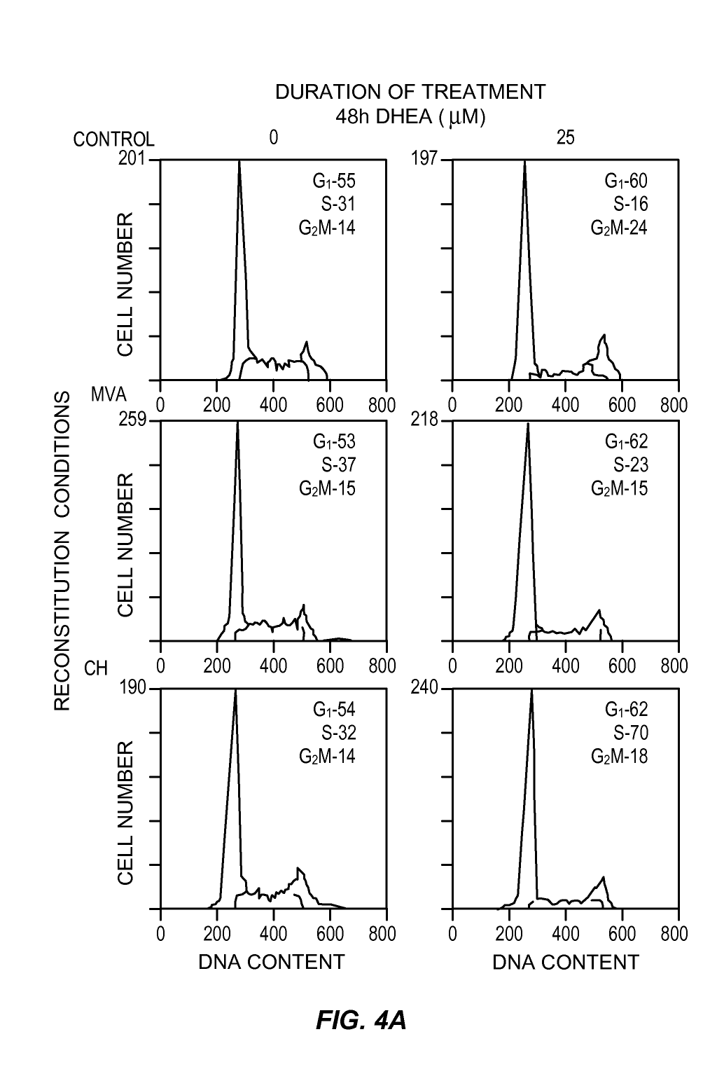

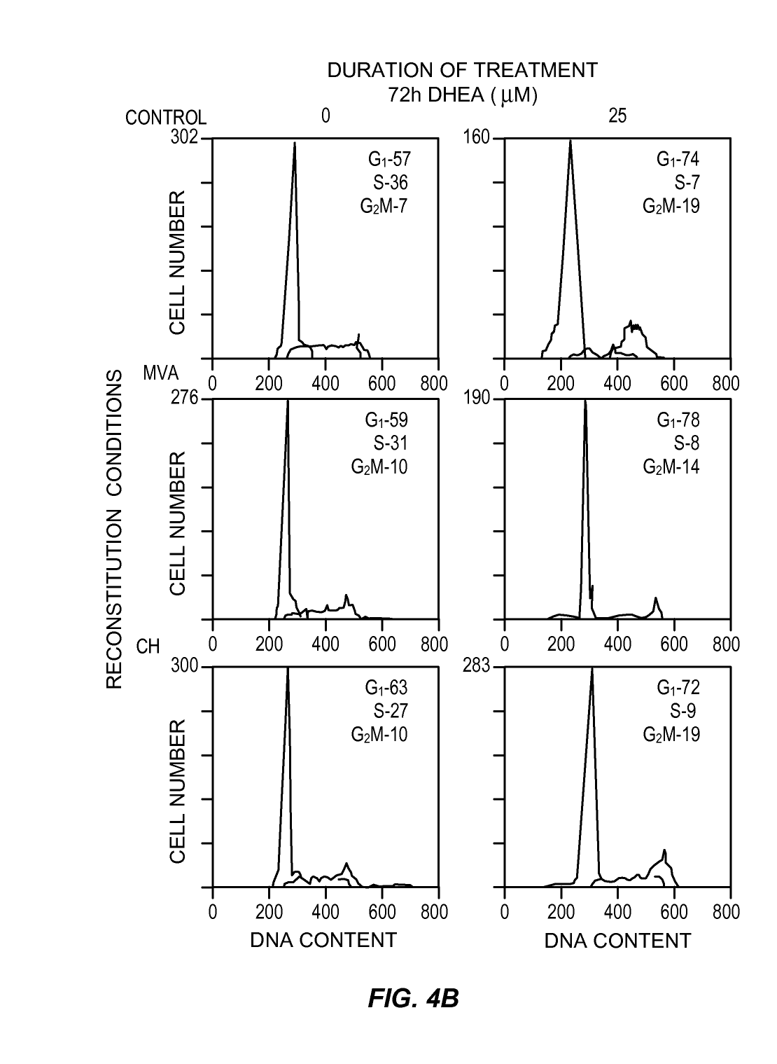

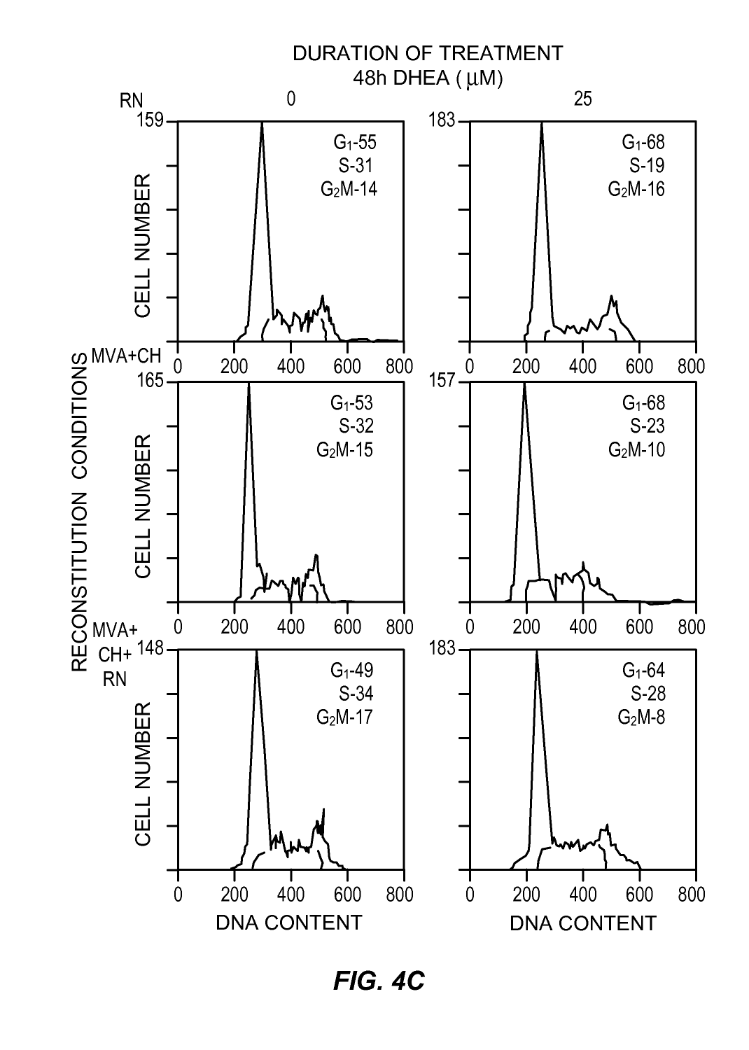

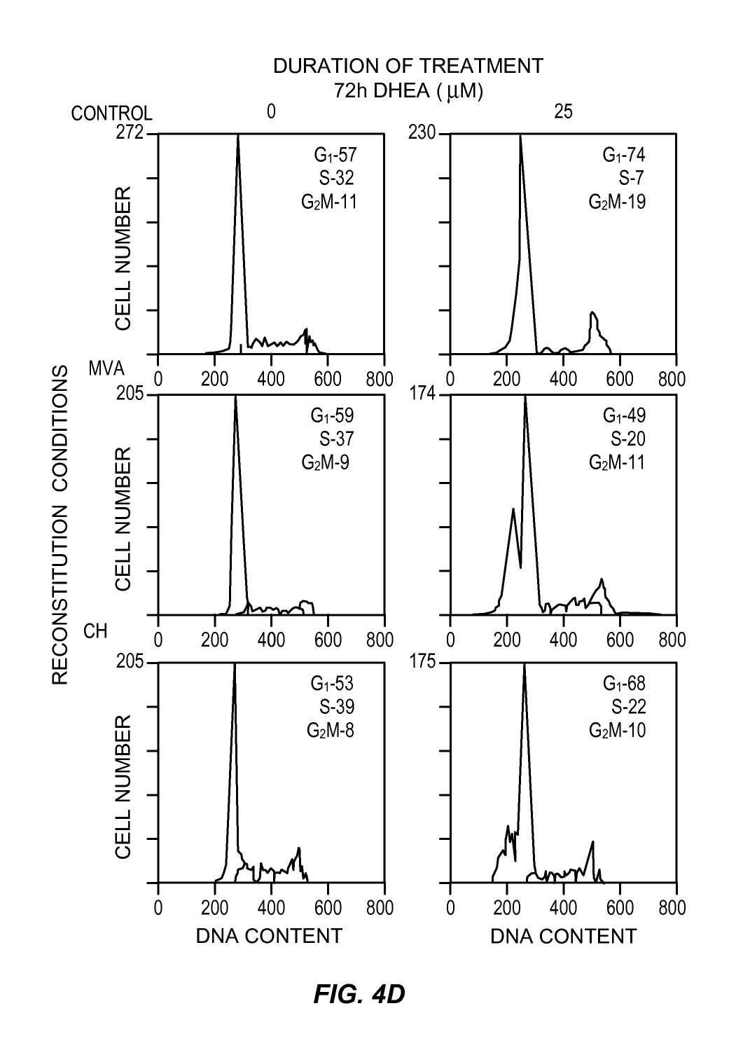

FIG. 4 illustrates reversal of DHEA-induced arrest in HT-29 SF cells. Cells were plated (10⁵ cells/60 mm dish) and 48 hours later treated with either 0 or 25 FM DHEA. The medium was supplemented with 2 FM MVA; 15 Fg/ml CH; a mixture of RN containing uridine, cytidine, adenosine, and guanosine in final concentrations of 30 FM; MVA plus CH (MVA+CH); or MVA plus CH plus RN (MVA+CH+RN) or was not supplemented. Cells were harvested after 48 or 72 hours, fixed in ethanol, and stained with propidium iodine, and the DNA content per cell was determined by flow cytometric analysis. The percentage of cells in G₁, S, and G₂M phases were calculated using the Cellfit cell cycle profile analysis program S phase is marked by a quadrangle for clarity. Representative histograms from duplicative determinations are shown. The experiment was repeated two times. Note that supplements had little effect on cell cycle progression in the absence of DHEA.

With increasing exposure time, DHEA progressively reduced the proportion of cells in S phase. While inclusion of MVA partially prevented this effect in the initial 48 hours but not after 72 hours, the addition of MVA plus CH was also able to partially prevent S phase depletion at 72 hours, suggesting a requirement of both MVA and CH for cell progression during prolonged exposure. The addition of MVA, CH, and RN was apparently most effective at reconstitution but still did not restore the percentage of S phase cells to the value seen in untreated control cultures. CH or RN alone had very little effect at 48 hours and no effect at 72 hours. Morphologically, cells responded to DHEA by acquiring a rounded shape, which was prevented only by the addition of MVA to the culture medium (data not shown). Some of the DNA histograms after 72 hours DHEA exposure in FIG. 4 also show the presence of a subpopulation of cells possessing apparently reduced DNA content. Since the HT-29 cell line is known to carry populations of cells containing varying numbers of chromosomes (68-72; ATCC), this may represent a subset of cells that have segregated carrying fewer chromosomes.

### Example 9

### Conclusions

The examples above provide evidence that in vitro exposure of HT-29 SF human colonic adenocarcinoma cells to concentrations of DHEA known to deplete endogenous mevalonate results in growth inhibition and G₁ arrest and that addition of MVA to the culture medium in part prevents these effects. DHEA produced effects upon protein isoprenylation which were in many respects similar to those observed for specific 3-hydroxy-3-methyl-glutaryl-CoA reductase inhibitors such as lovastatin and compactin. Unlike direct inhibitors of mevalonate biosynthesis, however, DHEA mediates its effects upon cell cycle progression and cell growth in a pleiotropic manner involving ribo- and deoxyribonucleotide biosynthesis and possibly other factors as well.

### Example 10

### Effect of CoQs & an EA on In Vitro NADPH Levels

Glocose-6Phosphate Dehydrogenase (G6PD) is an important enzyme that is widespread in mammals, and is involved in the conversion of NADP to NADPH, thereby increasing NADPH levels. An inhibition of the G6PD enzyme, thus, will be expected to result in a reduction of cellular NADPH levels, which event, in turn, will be expected to inhibit pathways that are heavily dependent on NADPH. One such pathway, the so-called One-Carbon-Pool pathway, also known as the Folate Pathway, is directly involved in the production of adenosine by addition of the C₂ and C₈ carbon atoms of the purine ring. Consequently, the inhibition of this pathway will lead to adenosine depletion.

The present invention is broadly applicable to Epiandrosterones (EAs) and Ubiquinones (CoQs). The description of the pathways involved in the present invention is described in the Background section. The present experiment was designed to show that one EA and two CoQs inhibit NADPH levels. DHEA, an Epiandrosterone, has already been shown to decrease levels of adenosine in various tissues. See, Examples 1 and 2 above. The fact that two CoQs are shown to lower NADPH levels to a similar extent as an Epiandrosterone, let alone to a similar extent ensures that the NADPH reduction caused by the CoQs will also result in lower cellular adenosine levels or in adenosine cell depletion. Thus, in accordance with the invention, both Epiandrosterones and Ubiquinones decrease levels of adenosine and, therefore, are useful as medicaments for use in the treatment of diseases where a decrease of adenosine levels or its depletion is desirable, including respiratory diseases such as asthma, bronchoconstriction, lung inflammation and allergies and the like. Both Ubiquiniones and DHEA inhibit NADPH levels in a statistically significant manner, when compared to a control. Moreover, the Ubiquinone inhibits NADPH levels to a similar extent as DHEA. The present invention is broadly applicable to the use of Epiandrosterones (EAs) and Ubiquinones (CoQs) to the treatment of respiratory and lung diseases, and other diseases associated with varying levels of adenosine, adenosine hypersensitivity, asthma, bronchoconstriction, and/or lung inflammation and allergies. The DHEA and Ubiquinones employed in the present experiments are equivalent to those described and exemplified above.

Enzymatic Assay of Purified G6PDH

The reaction mixture contained 50 mM glycyl glycine buffer, pH 7.4, 2 mM D-glucose-6-phosphate, 0.67 mM Beta-NADP, 10 mM MgCL2 and 0.0125 units of G6PDH in a final volume of 3.0 ml. All experiments were repeated 4 times.

The control group contained 3 samples that were added no DHEA or Ubiquinone. The experimental group contained a similar number of samples (3) for each concentration of DHEA or Ubiquinone. One group was added DHEA (in triplicate) at different concentrations. A second group was added different concentrations of a CoQ of long side chain (in triplicate), and a third group received a CoQ of short side chain (in triplicate), both at various doses in the μM range.

The reaction was started by addition of the enzyme, and the increase in absorbance at 340 nm was measured for 5 minutes. Each data point was conducted in triplicate, and the fill experiment was repeated 4 times.

Both DHEA and the Ubiquinones inhibited the enzyme activity in a statistically significant manner when compared to controls. DHEA was found to inhibit by 72% in vitro the activity of purified G6PDH when compared to control. Both Ubiquinones inhibited the activity of purified G6PDH in vitro by an amount that was not statistically significantly different from that of DHEA. Both DHEA and the Ubiquinones inhibited the enzyme in a statistically significant manner when compared to controls. Both long chain and short chain CoQs were found to be effective inhibitors of G6PDH.

The above results clearly indicate that CoQ reduced cellular levels of NADPH to an extent similar to DHEA and consequently cellular adenosine levels, and has a therapeutic effect on diseases and conditions associated with them. The present results show that CoQs have a therapeutic effect similar to that of epiandrosterones. The pathways involved in the present invention, as described above, show the criticality of the results reported here, showing that an Epiandrosterone (DHEA) and tow Ubiquinones inhibit NADPH levels in a statistically significant manner. The same epiandrosterone (DHEA) was shown in Examples 1 and 2 to decrease levels of adenosine in various tissues. The two different Ubiquinones employed lowered NADPH levels to a similar extent as DHEA. The NADPH reduction caused by the Ubiquinones will, in the case of DHEA, result in lower cellular adenosine levels or adenosine depletion. Thus, in accordance with the invention, both Epiandrosterones and Ubiquinones decrease levels of adenosine and are, therefore, useful in the therapy of diseases and conditions where a decrease of adenosine levels or its depletion are desirable, including respiratory and airway diseases such as asthma, bronchoconstriction, lung inflammation and allergies, and the like.

These are clearly superior results, which could not have been expected based on the knowledge of the art at the time of this invention. The experimental data and results provided are clearly enabling of the effect of Ubiquinones on adenosine cellular levels and, therefore, on its therapeutic affect on diseases and conditions associated with them, as described and claimed in this patent.

In Examples 11 to 16 micronized DHEA or Ubiquinone and micronized salmeterol (as the hydroxynaphthoate) are added in the proportions given below either dry or after predispersal in a small quantity of stabilizer, disodium dioctylsulphosuccinate, lecithin, oleic acid or sorbitan trioleate/trichloro-fluoromethane solution to a suspension vessel containing the main bulk of the trichlorofluoromethane solution. The resulting suspension is further dispersed by an appropriate mixing system using, for example, a high shear blender, ultrasonics or a microfluidiser until an ultrafine dispersion is created. The suspension is then continuously recirculated to suitable filling equipment designed for cold fill or pressure filling of dichlorodifluoromethane. The suspension may be also prepared in a suitable chilled solution of stabilizer, in trichlorofluoromethane/dichloro-difluoromethane.

### Example 11

### Metered Dose Inhaler

| | Target per | |
|---|---|---|
| Active Ingredient | Actuation | |
| Salmeterol | 25.0 | μg |
| (as hydroxynaphthoate) | | |
| DHEA | 400 | mg |
| Stabilizer | 5.0 | μg |
| Trichlorofluoromethane | 23.70 | mg |
| Dichlorodifluoromethane | 61.25 | mg |

### Example 12

### Metered Dose Inhaler

| | Target per | |
|---|---|---|
| Active Ingredient | Actuation | |
| Salmeterol | 25.0 | μg |
| (as hydroxynaphthoate) | | |
| DHEA-S | 400 | mg |
| Stabilizer | 7.5 | μg |
| Trichlorofluoromethane | 23.67 | mg |
| Dichlorodifluoromethane | 61.25 | mg |

### Example 13

### Metered Dose Inhaler

| | Target per | |
|---|---|---|
| Active Ingredient | Actuation | |
| Salmeterol | 25.0 | μg |
| (as hydroxynaphthoate) | | |
| Ubiquinone | 400 | mg |
| Stabilizer | 25.0 | μg |
| Trichlorofluoromethane | 23.45 | mg |
| Dichlorodifluoromethane | 61.25 | mg |

### Example 14

### Metered Dose Inhaler

| | Target per | |
|---|---|---|
| Active Ingredient | Actuation | |
| Albuterol | 25.0 | μg |
| (as hydroxynaphthoate) | | |
| DHEA | 400.0 | mg |
| Stabilizer | 15.0 | μg |
| Trichlorofluoromethane | 23.56 | mg |
| Dichlorodifluoromethane | 61.25 | mg |

### Example 15

### Metered Dose Inhaler

| | Target per | |
|---|---|---|
| Active Ingredient | Actuation | |
| Albuterol | 25.0 | μg |
| (as hydroxynaphthoate) | | |
| DHEA-S | 400.0 | mg |
| Stabilizer | 15.0 | μg |
| Trichlorofluoromethane | 23.56 | mg |
| Dichlorodifluoromethane | 61.25 | mg |

### Example 16

### Metered Dose Inhaler

| | Target per | |
|---|---|---|
| Active Ingredient | Actuation | |
| Albuterol | 100.0 | μg |
| (as hydroxynaphthoate) | | |
| Ubiquinone (CoQ₁₀) | 400.0 | mg |
| Stabilizer | 25.0 | μg |
| Trichlorofluoromethane | 23.43 | mg |
| Dichlorodifluoromethane | 61.25 | mg |

In the following Examples 17 to 22, the active ingredients are micronized and bulk blended with lactose in the proportions given above. The blend is filled into hard gelatin capsules or cartridges or into specifically constructed double foil blister packs (Rotadisks blister packs, Glaxo® to be administered by an inhaler such as the Rotahaler inhaler (Glaxo®) or in the case of the blister packs with the Diskhaler inhaler (Glaxo®).

### Example 17

### Metered Dose Dry Powder Formulation

| Active Ingredient | /cartridge or blister | |
|---|---|---|
| Salmeterol (hydroxynaphthoate) | 72.5 | μg |
| DHEA | 1.00 | mg |
| Lactose Ph. Eur. to | 12.5 or 25.0 | mg |

### Example 18

### Metered Dose Dry Powder Formulation

| Active Ingredient | /cartridge or blister | |
|---|---|---|
| Albuterol (hydroxynaphthoate) | 72.5 | μg |
| DHEA-S | 1. | mg |
| Lactose Ph. Eur. to | 12.5 or 25.0 | mg |

### Example 19

### Metered Dose Dry Powder Formulation

| Active Ingredient | /cartridge or blister | |
|---|---|---|
| Albuterol (hydroxynaphthoate) | 72.5 | μg |
| Ubiquinone (CoQ₁₀) | 1 | mg |
| Lactose Ph. Eur. to | 12.5 or 25.0 | mg |

### Example 20

### Metered Dose Dry Powder Formulation

| Active Ingredient | /cartridge or blister | |
|---|---|---|
| Salmeterol (hydroxynaphthoate) | 72.5 | μg |
| DHEA | 1 | mg |
| Lactose Ph. Eur. to | 12.5 or 25.0 | mg |

### Example 21

### Metered Dose Dry Powder Formulation

| Active Ingredient | /cartridge or blister | |
|---|---|---|
| Salmeterol (hydroxynaphthoate) | 72.5 | μg |
| DHEA-S | 1 | mg |
| Lactose Ph. Eur. to | 12.5 or 25.0 | mg |

### Example 22

### Metered Dose Dry Powder Formulation

| Active Ingredient | /cartridge or blister | |
|---|---|---|
| Salmeterol (hydroxynaphthoate) | 145.0 | μg |
| Ubiquinone (CoQ₁₀) | 1 | mg |
| Lactose Ph. Eur. to | 12.5 or 25.0 | mg |

The foregoing examples are illustrative of the present invention, but should not to be construed as limiting thereof. The invention is defined by the following claims, with equivalents of the claims to be included therein.

What is claimed is:

1. A pharmaceutical composition, comprising a pharmaceutically or veterinarily acceptable carrier and amounts of the first and second active agents effective to treat a respiratory or malignant lung disease, wherein the first active agent is dehydroepiandrosterone (DHEA) or dehydroepiandrosterone sulfate (DHEA-S); and the second active agent is a bronchodilating agent; and wherein the composition, when administered to a subject, contains particles of a size sufficiently small to pass through mouth and larynx upon inhalation and continue into bronchi and alveoli of lungs.

2. The composition of claim 1, wherein the first active agent is a DHEA-S that has a counter ion, wherein the counter ion comprises H, sodium, potassium, magnesium, aluminum, zinc, calcium, lithium, or ammonium.

3. The composition of claim 1, wherein the second active agent is selected from β2 adrenergic agonists, anti-cholinergic agents, anti-histaminic agents, adenosine receptor antagonists or glucocorticosteroids.

4. The composition of claim 1, wherein the second active agent comprises a β2 adrenergic agonist selected from ephedrine, isoproterenol, isoetharine, epinephrine, metaproterenol, terbutaline, fenoterol, procaterol, albuterol, salbutamol, pirbuterol, formoterol, biloterol, bambuterol, salmeterol or seretide.

5. The composition of claim 1, wherein the second active agent comprises a glucocorticosteroid.

6. The composition of claim 1, wherein the second active agent comprises an anti-cholinergic agent.

7. The composition of claim 6, wherein the anti-cholinergic agent is selected from the group consisting of ipratropium bromide, oxitropium bromide, and tiotropium.

8. The composition of claim 1, wherein the second active agent comprises a leukotriene inhibitor.

9. The composition of claim 1, wherein the second active agent comprises a mucolytics.

10. The composition of claim 1, wherein the second active agent comprises a methylxanthine.

11. The composition of claim 10, wherein the methylxanthine is aminophylline or theophylline.

12. The composition of claim 1, further comprising a ubiquinone or pharmaceutically or veterinarily acceptable salt thereof, wherein the ubiquinone has the chemical formula 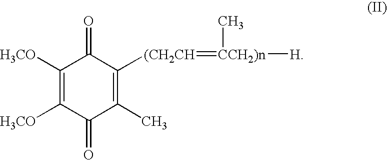
wherein n is 1 to 12.

13. The composition of claim 1, further comprising an agent selected from other therapeutic or bioactive agents, preservatives, anti-oxidants, isotonic agents, flavoring agents, volatile oils, buffering agents, dispersants or surfactants.

14. The composition of claim 13, wherein the other therapeutic or bioactive agents are selected from the group consisting of analgesics, pre-menstrual medications, menopausal agents, anti-aging agents, anti-anxyolytic agents, mood disorder agents, anti-depressants, anti-bipolar mood agents, anti-schyzophrenic agents, anti-cancer agents, alkaloids, blood pressure controlling agents, hormones, anti-inflammatory agents, muscle relaxants, steroids, soporific agents, anti-ischemic agents, anti-arrythmic agents, contraceptives, vitamins, minerals, tranquilizers, neurotransmitter regulating agents, wound healing agents, anti-angyogenic agents, cytokines, growth factors, anti-metastatic agents, antacids, anti-histaminic agents, anti-bacterial agents, anti-viral agents, anti-gas agents, appetite suppressants, sun screens, emollients, skin temperature lowering products, radioactive phosphorescent or fluorescent contrast diagnostic or imaging agents, libido altering agents, bile acids, laxatives, anti-diarrheic agents, skin renewal agents, hair growth agents, analgesics, pre-menstrual medications, anti-menopausal agents, hormones, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, hormones, anti-inflammatory agents, arthritis, burns, wounds, chronic bronchitis, chronic obstructive pulmonary disease (COPD), inflammatory bowel disease such as Crohn's disease, ulcerative colitis, autoimmune disease, lupus erythematosus, muscle relaxants, steroids, soporific agents, anti-ischemic agents, anti-arrhythmic agents, contraceptives, vitamins, minerals, tranquilizers, neurotransmitter regulating agents, wound and burn healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent or fluorescent contrast diagnostic or imaging agents, libido altering agents, bile acids, laxatives, anti-diarrheic agents, skin renewal agents or hair growth agents.

15. The composition of claim 1, which is a systemic or topical formulation, and wherein the carrier comprises a gaseous, solid or liquid carrier.

16. The formulation of claim 15, wherein the formulation is an oral, nasal, inhalable, topical, parenteral or transdermal formulation.

17. The formulation of claim 16, wherein the formulation is an oral, intrabuccal, sub-lingual, intrapulmonary, respirable, inhalable, nasal, rectal, intrauterine, vaginal, intratumor, intracranial, subcutaneous, intravascular, sublingual, intravenous, intrathecal, transdermal, intradermal, intracavitary, implantable, iontophoretic, intraocular, ophthalmic, intraarticular, otical, intravenous, intramuscular, intraglandular, intraorgan, intralymphatic, slow release or enteric coating formulation.

18. The formulation of claim 16, wherein the formulation is an oral formulation.

19. The formulation of claim 18, wherein the oral formulation is selected from the group consisting of capsules, cachets, lozenges, tablets, powder, granules, solutions, suspensions or emulsions.

20. The formulation of claim 18, further comprising an enteric coating.

21. The formulation of claim 16, wherein the formulation is a solution, suspension or emulsion selected from the group consisting of aqueous or non-aqueous liquid solutions or suspensions, or oil-in-water or water-in-oil emulsions.

22. The formulation of claim 16, which is a topical formulation.

23. The formulation of claim 22, wherein the formulation is selected from the group consisting of ointments, creams, lotions, pastes, gels, sprays, aerosols or oils; and may further comprise a carrier selected from vaseline, lanoline, polyethylene glycols, alcohols or trans-dermal enhancers.

24. The formulation of claim 16, wherein the formulation is a transdermal formulation.

25. The formulation of claim 24, wherein the formulation is in the form of a patch or an iontophoretic formulation.

26. The formulation of claim 16, wherein the formulation is a nasal, inhalable, respirable, intrapulmory or intratracheal formulation.

27. The formulation of claim 26, wherein the formulation is an aerosol or spray comprising liquid or solid powdered particles.

28. The composition of claim 1, wherein the particles are about 0.05 μm to about 10 μm in size.

29. The composition of claim 1, wherein the particles are about 0.5 μm to about 5 μm in size.

30. The composition of claim 1, wherein the particles are about 8 μm to about 500 μm in size.

31. The composition of claim 1, wherein the composition is given in bulk or in single- or multi-dose form.

32. The composition of claim 31, wherein the composition is provided in sealed ampoules, vials, cartridges or blisters.

33. The composition of claim 1, wherein the composition is freeze-dried or lyophilized.

34. A kit, comprising a delivery device, and the formulation of claim 26.

35. The kit of claim 34, wherein the delivery device comprises an aerosol or spray generator.

36. The kit of claim 35, wherein the aerosol generator comprises an inhaler.

37. The kit of claim 36, wherein the inhaler delivers individual pre-metered doses of the formulation

38. The kit of claim 36, wherein the inhaler comprises a nebulizer or insufflator.

39. The kit of claim 34, wherein the delivery device comprises a compression inhaler, and the formulation comprises a suspension or solution in an aqueous, or non-aqueous liquid, or an oil-in-water, or water-in-oil emulsion.

40. The kit of claim 34, wherein the formulation is provided in a capsule, cartridge or blister, which may be a pierceable or openable capsule, cartridge or blister.

41. The kit of claim 34, wherein the delivery device is pressurized and it operates with the aid of a propellant.

42. A method for treatment of a respiratory or malignant lung disorder or condition, or for reducing levels of, or sensitivity to, adenosine or adenosine receptors, or for increasing surfactant or ubiquinone levels in a subject in need of treatment, comprising simultaneously, sequentially or separately administering to a subject in need of treatment prophylactically or therapeutically effective amounts of the first and second active agents of claim 1.

43. The method of claim 42, wherein the disorder or condition comprises asthma, chronic obstructive pulmonary disease (COPD), cystic fibrosis (CF), dyspnea, emphysema, wheezing, pulmonary hypertension, pulmonary fibrosis, hyper-responsive airways, increased adenosine or adenosine receptor levels, adenosine hypersensitivity, infectious diseases, pulmonary bronchoconstriction, respiratory tract inflammation or allergies, lung surfactant or ubiquinone depletion, chronic bronchitis, bronchoconstriction, difficult breathing, impeded or obstructed lung airways, adenosine test for cardiac function, pulmonary vasoconstriction, impeded respiration, Acute Respiratory Distress Syndrome (ARDS), administration of adenosine or adenosine level increasing drugs, infantile Respiratory Distress Syndrome (infantile RDS), pain, allergic rhinitis, cancer, chronic bronchitis, asbestosis and silicosis.

44. The method of claim 42, wherein the first active agent comprises dehydroepiandrosterone (DHEA) or dehydroepiandrosterone sulfate (DHEA-S), and is administered in an amount of about 0.05 to about 2000 mg/kg body weight/day.

45. The method of claim 42, further comprises a ubiquinone of formula (II) or salt thereof, and it is administered in an amount of about 1 to 150 mg/kg body weight/day. 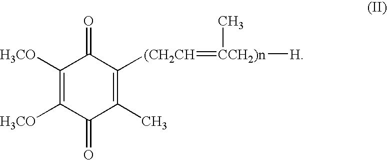
wherein n is 1-12.

46. The method of claim 42, wherein the respiratory or lung disease or condition is associated with an infectious disease, respiratory tract allergies or surfactant depletion.

47. The method of claim 42, wherein the disorder or condition comprises COPD.

48. The method of claim 42, wherein the disorder or condition comprises asthma.

49. The method of claim 42, wherein the disorder or condition comprises bronchoconstriction, wheezing, difficulty breathing or hypoxia.

50. The method of claim 42, wherein the first active agent comprises DHEA or DHEA-S and ubiquinone, and the second active agent comprises a β2 adrenergic agonist selected from the group consisting of ephedrine, isoproterenol, isoetharine, epinephrine, metaproterenol, terbutaline, fenoterol, procaterol, albuterol, salbutamol, pirbuterol, formoterol, biloterol, bambuterol, salmeterol and seretide.

51. The method of claim 42, wherein the first active agent comprises DHEA or DHEA-S and ubiquinone, and the second active agent comprises an anti-cholinergic agent selected from the group consisting of ipratropium bromide, oxitropium bromide, and tiotropium.

52. The method of claim 42, wherein the first active agent comprises DHEA or DHEA-S and ubiquinone, and the second active agent comprises a methylxanthine selected from the group consisting of aminophylline or theophylline.

53. The method of claim 42, wherein the subject is a human or a non-human animal.

54. The method of claim 42, which is a prophylactic or a therapeutic method.

55. The method of claim 42, wherein the respiratory disease comprises bronchoconstriction, lung inflammation or allergies, decreased lung surfactant or decreased ubiquinone, or DHEA or DHEA-S levels.

56. The method of claim 42, wherein the first active agent comprises DHEA or DHEA-S and is administered in an amount of about 1 to about 200/mg/kg/day; and the second active agent comprises salmeterol or a salt thereof, and is administered in an amount about 25 to about 500 pg per day.

57. The method of claim 42, wherein the first agent further comprises a ubiquinone of formula (II), or salt thereof, and it is administered in an amount about 0.1 to about 1200 mg/kg/day; and the second active agent comprises salmeterol or a salt thereof, and is administered in an amount about 25 to about 500 μg per day.

58. The method of claim 42, wherein the first and second active agents are administered by inhalation, into the airways of respiration, intrapulmonarily, nasally, orally, bucally, rectally, vaginally, into a tumor or fibroma, parenterally, sublingually, transdermally, topically, iontophorically, intracavitarily, by implant, sub-lingually, opthalmically, otically, intraarticularly, intralymphatically, by slow or sustained release or interically coated.

59. The method of claim 58, wherein the agents are administered nasally, intrapulmonarily, by inhalation or into the respiratory airways.

60. The method of claim 59, wherein the agents are administered as a liquid or powdered aerosol or spray of particle size of about 0.05 to about 50 μm.
